(19) United States
(12) Patent Application Publication
(10) Pub. No.: US 2023/0112958 A1
(43) Pub. Date: Apr. 13, 2023

(54) Smart Drug Delivery System and Pharmaceutical Kit for Dual Nuclear Medical Cytotoxic Theranostics

(71) Applicant: SCV-SpezialChemikalienVertrieb GmbH, Berlin (DE)

(72) Inventors: Frank RÖSCH, Berlin (DE); Hanane LAHNIF, Gau-Algesheim (DE); Tilmann GRUS, Mainz (DE)

(73) Assignee: SCV-SpezialChemikalienVertrieb GmbH, Berlin (DE)

(21) Appl. No.: 17/786,844

(22) PCT Filed: Dec. 17, 2020
(86) PCT No.: PCT/EP2020/086811

§ 371 Date: Jun. 17, 2022

(30) Foreign Application Priority Data
Dec. 20, 2019 (DE) 10 2019 135 564.8

(51) Int. Cl. A61K 51/08; A61K 47/64

(52) U.S. Cl. CPC A61K 51/088; A61K 47/64

(57) ABSTRACT

The invention generally relates to a smart drug delivery system for dual nuclear medical cytotoxic theranostics incorporating either (i) a first compound with the structure CT-L1-Chel-S1-TV or

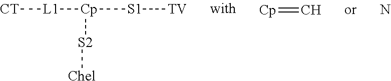

or (ii) a second compound with the structure Chel-S-TV and a third compound with the structure CT-L-TV. In the first, second and third compounds Chel is a radical of a chelating agent for complexing a radioisotope; CT is a radical of a cytotoxic compound; TV is a biological targeting vector; L1 and L are each linkers; S1, S2 and S are each spacers.

The present invention relates to a smart drug delivery system and to a pharmaceutical kit for dual nuclear-medical/cytotoxic theranostics.

The smart drug delivery system comprises
a first compound having the structure

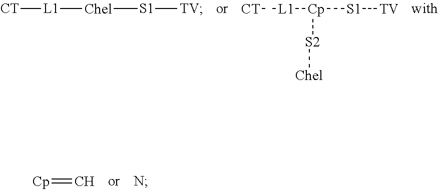

or
a second compound having the structure Chel-S-TV and a third compound having the structure CT-L-TV;

wherein, in the first, second and third compounds,

Chel is a radical of a chelator for the complexation of a radioisotope; CT is a radical of a cytotoxic compound; TV is a biological targeting vector; L1 and L are each a linker; S1, S2 and S are each a spacer.

The pharmaceutical kit consists of
a first vessel containing a first compound or a first carrier substance containing the first compound;

or
a second vessel containing a second compound or a second carrier substance containing the second compound; and
a third vessel containing a third compound or a third carrier substance containing the third compound;

wherein

the first compound has the structure

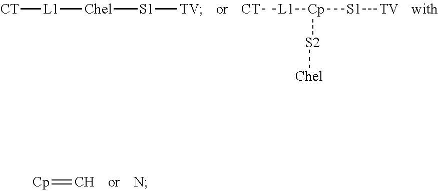

the second compound has the structure Chel-S-TV;

and the third compound has the structure CT-L-TV;

in which

Chel is a radical of a chelator for the complexation of a radioisotope; CT is a radical of a cytotoxic compound; TV is a biological targeting vector; L1 and L are each a linker; S1, S2 and S are each a spacer.

Cytotoxic pharmaceuticals, for example doxorubicin, have been used for decades in chemotherapy. In conventional systemic chemotherapy, the cytotoxic pharmaceutical is administered intravenously, orally or peritoneally in relatively high dose. As well as cancer cells, cytotoxic pharmaceuticals also damage healthy tissue, especially cells having a high division rate, and cause severe side effects, some of them life-threatening, which frequently force treatment to be discontinued.

In order to alleviate side effects, low-dose targeted cytotoxic pharmaceuticals having high binding affinity to tumor cells have been used for a few years. Tumor affinity is mediated by targeting vectors conjugated to the cytotoxic active ingredient. Targeting vectors are generally agonists (substrates) or antagonists (inhibitors) of membrane-bound proteins that are significantly overexpressed on the envelope of tumor cells compared to healthy body cells. Targeting vectors include simple organic compounds, oligopeptides having natural or derivatized amino acids, and aptamers.

Moreover, imaging nuclear-medical diagnosis methods have been used to an increasing degree in clinical treatment for about 15 years, such as positron emission tomography (PET) and single-photon emission computed tomography (SPECT). Theranostic methods have recently also been gaining in significance.

Imaging nuclear-medical diagnosis and treatment (theranostics) of cancers assists and supplements chemotherapy.

In nuclear-medical diagnostics and theranostics, tumor cells are labeled or irradiated with a radioactive isotope, for example ⁶⁸Ga or ¹⁷⁷Lu. This involves using labeling precursors that bind the respective radioisotope covalently (¹⁸F) or coordinatively (⁶⁸Ga, ^{99m}Tc, ¹⁷⁷Lu). The labeling precursors, in the case of medical isotopes, comprise a chelator as an essential chemical component for the effective and stable complexation of the radioisotope, and a biological targeting vector as functional component that binds to target structures in the tumor tissue, especially membrane-bound proteins.

Targeting vectors having high affinity for cancer cells are equally suitable for targeted chemotherapy and for nuclear-medical diagnostics and theranostics. Accordingly, research in these disciplines is complementary.

After intravenous injection into the blood circulation, a nuclear-medical labeling precursor complexed with a radioisotope accumulates on or in tumor cells. In order to minimize the radiation dose in healthy tissue, a small amount of a radioisotope having a short half life of a few hours to days is used in diagnostic examinations.

The chelator modifies the configuration and chemical properties of the targeting vector and generally significantly affects its affinity to tumor cells. Accordingly, the coupling between the chelator and the at least one targeting vector is tailored in complex trial-and-error tests or what are called biochemical screenings. This involves synthesizing a large number of labeling precursors comprising the chelator and a targeting vector, and in particular quantifying the affinity for tumor cells. The chelate and the chemical coupling to the targeting vector are crucial to the biological and nuclear-medical potency of the respective labeling precursor.

In addition to high affinity, the labeling precursor must fulfill further requirements, such as
rapid and effective complexation or covalent binding of the respective radioisotope;
high selectivity for tumor cells relative to healthy tissue;
in vivo stability, i.e. biochemical stability in blood serum under physiological conditions.

Prostate Cancer

For men in developed countries, prostate cancer is the most common type of cancer and the third most lethal form of cancer. In this disease tumor growth proceeds only gradually, and the 5-year survival rate in the case of diagnosis at an early stage is nearly 100%. If the cancer is only discovered when the tumor has metastasized, the survival rate falls dramatically. Too early and too aggressive action against the tumor, on the other hand, can unnecessarily impair the patient's quality of life. For example, the surgical removal of the prostate can lead to incontinence and impotence. Reliable diagnosis and information as to the stage of the disease are essential for a successful treatment with a high quality of life for the patient. A common means of diagnosis, aside from prostate palpation by a doctor, is the determination of tumor markers in the patient's blood. The most prominent marker for prostate carcinoma is the concentration of the prostate-specific antigen (PSA) in the blood. However, the significance of PSA concentration is disputed, since patients having slightly elevated values often do not have prostate carcinoma, but 15% of patients with prostate carcinoma do not show an elevated PSA concentration in the blood. A further target structure for the diagnosis of prostate tumors is the prostate-specific membrane antigen (PSMA). By contrast with PSA, PSMA cannot be detected in the blood. It is a membrane-bound glycoprotein having enzymatic activity. Its function is the elimination of C-terminal glutamate from N-acetyl-aspartyl-glutamate (NAAG) and folic acid-(poly)-γ-glutamate. PSMA barely occurs in normal tissue, but is significantly overexpressed by prostate carcinoma cells, with close correlation of the expression with the tumor stage. Lymph node and bone metastases of prostate carcinomas also show expression of PSMA to an extent of 40%.

One strategy in the molecular targeting of PSMA consists in binding with antibodies to the protein structure of the PSMA. A further approach is to utilize the enzymatic activity of PSMA, which is well understood. In the enzymatic binding pocket of PSMA there are two Zn²⁺ ions that bind glutamate. An aromatic binding pocket is situated in front of the center with the two Zn²⁺ ions. The protein is capable of expanding to accommodate a binding partner (induced fit), such that it can bind not only NAAG but also folic acid, with the pteroic acid group docking within the aromatic binding pocket. The utilization of the enzymatic affinity of PSMA enables the uptake of the substrate into the cell (endocytosis) irrespective of any enzymatic cleavage of the substrate.

Therefore, PSMA inhibitors are particularly suited as targeting vectors for imaging diagnostic and theranostic radiopharmaceuticals or radiotracers. The radiolabeled inhibitors bind to the active site of the enzyme, but are not converted there. The binding between the inhibitor and the radioactive label is thus not parted. Promoted by endocytosis, the inhibitor with the radioactive label is internalized into the cell and accumulates in tumor cells.

Inhibitors possessing high affinity to PSMA (scheme 1) generally contain a glutamate motif and an enzymatically non-cleavable structure. A highly effective PSMA inhibitor is 2-phosphonomethylglutaric acid or 2-phosphonomethyl-pentanedioic acid (2-PMPA), in which the glutamate motif is bound to a phosphonate group which is not cleavable by PSMA. A further group of PSMA inhibitors which is utilized in the clinically relevant radiopharmaceuticals PSMA-11 (scheme 2) and PSMA-617 (scheme 3) is that of urea-based inhibitors.

It has proven advantageous to address the aromatic binding pocket of PSMA in addition to the binding pocket for the glutamate motif. For example, in the highly effective radiopharmaceutical PSMA-11, the L-lysine-urea-L-glutamate (KuE) binding motif is bound via hexyl (hexyl linker) to an aromatic HBED chelator (N,N′-bis(2-hydroxy-5-(ethylene-beta-carboxy) benzyl)ethylenediamine N,N′-diacetate).

If L-lysine-urea-L-glutamate (KuE), by contrast, is bound to the nonaromatic chelator DOTA (1,4,7,10-tetraazacyclododecane-1,4,7,10-tetraacetate), reduced affinity and accumulation in tumor tissue is found. Nevertheless, in order to utilize the DOTA chelator for a radiopharmaceutical with PSMA affinity and therapeutic radioisotopes, such as ¹⁷⁷Lu or ²²⁵Ac, the linker has to be adapted. By means of specific replacement of hexyl by various aromatic structures, the highly effective radiopharmaceutical PSMA-617, the current gold standard, was found.

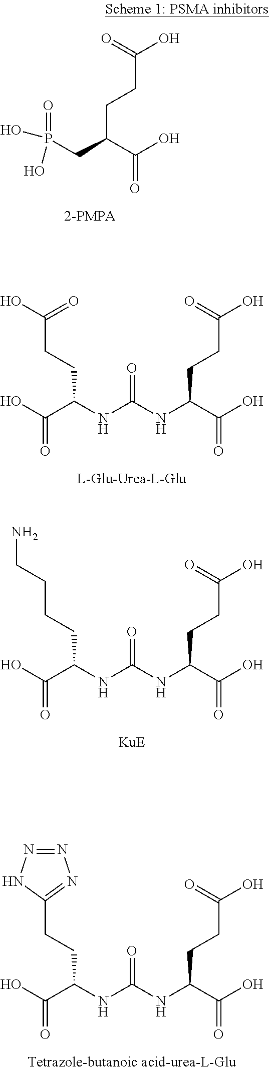 [SMILES: O=C(O)CC[C@H](CP(=O)(O)O)C(=O)O ; O=C(O)CC[C@H](NC(=O)N[C@@H](CCC(=O)O)C(=O)O)C(=O)O ; NCCCC[C@H](NC(=O)N[C@@H](CCC(=O)O)C(=O)O)C(=O)O ; O=C(O)CC[C@H](NC(=O)N[C@@H](CCc1nnn[nH]1)C(=O)O)C(=O)O]

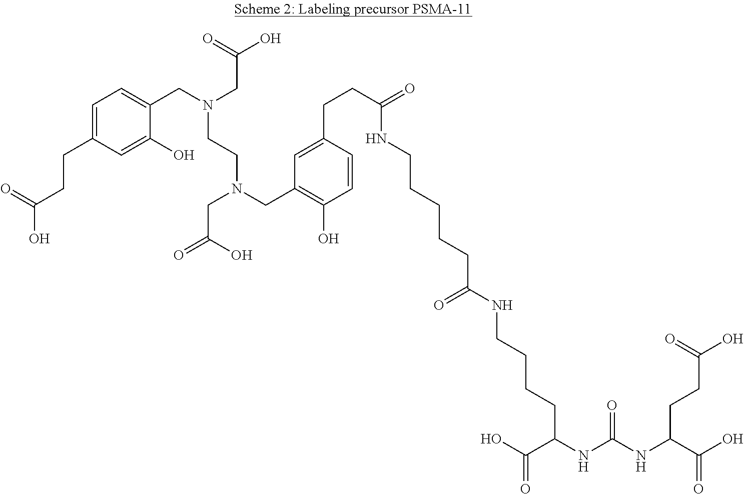 [SMILES: O=C(O)CCc1ccc(CN(CCN(CC(=O)O)Cc2cc(CCC(=O)NCCCCCC(=O)NCCCCC(NC(=O)NC(CCC(=O)O)C(=O)O)C(=O)O)ccc2O)CC(=O)O)c(O)c1]

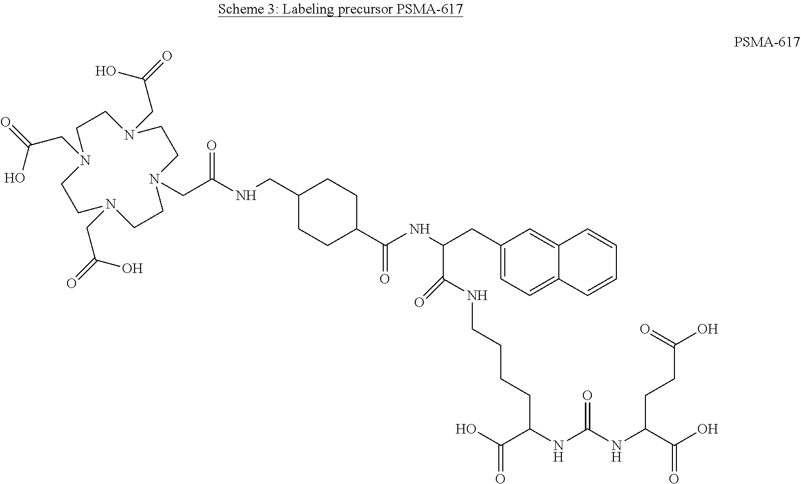 [SMILES: O=C(O)CCC(NC(=O)NC(CCCCNC(=O)C(Cc1ccc2ccccc2c1)NC(=O)C1CCC(CNC(=O)CN2CCN(CC(=O)O)CCN(CC(=O)O)CCN(CC(=O)O)CC2)CC1)C(=O)O)C(=O)O]

Tumor Stroma

Many tumors comprise malignant epithelial cells and are surrounded by multiple non-carcinogenic cell populations, including activated fibroblasts, endothelial cells, pericytes, immunoregulatory cells and cytokines in the extracellular matrix. These so-called stroma cells that surround the tumor play an important role in the development, growth and metastasis of carcinomas. A major portion of the stroma cells are activated fibroblasts, which are referred to as cancer-associated fibroblasts (CAFs). In the course of tumor progression, CAFs alter their morphology and biological function. These alterations are induced by intercellular communication between cancer cells and CAFs. CAFs here form a microenvironment that promotes cancer cell growth. It has been shown that therapies aimed solely at cancer cells are inadequate. Effective therapies must include the tumor microenvironment, i.e. CAFs. In more than 90% of all human carcinomas, CAFs overexpress fibroblast activation protein (FAP). Therefore, FAP represents a promising point of attack for nuclear-medical diagnosis and theranostics. Analogously to PSMA—especially FAP inhibitors (FAPI or FAPi) are suitable affine biological targeting vectors for FAP labeling precursors. FAP exhibits bimodal activity of dipeptidylpeptidase (DPP) and prolyloligopeptidase (PREP) that is catalyzed by the same active site. Accordingly, there are two possible types of inhibitors that inhibit the DPP activity and/or the PREP activity of FAP. Known inhibitors for the PREP activity of FAP have a low selectivity for FAP. In cancer types where both FAP and PREP are overexpressed, however, PREP inhibitors may also be suitable as targeting vectors in spite of their low FAP selectivity.

Scheme 4 shows a DOTA-conjugated FAP labeling precursor in which the chelator is coupled to the pharmacophore unit ((S)—N-(2-(2-cyano-4,4-difluoropyrrolidin-1-yl)-2-oxoethyl)-6-(4-aminobutyloxy)-quinoline-4-carboxamide via the 4-aminobutoxy functionality to the quinoline.

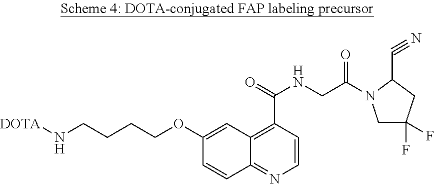

Bone Metastases

Bone metastases express farnesyl pyrophosphate synthase (FPPS), an enzyme in the HMG-CoA reductase (mevalonate) pathway. The inhibition of FPPS suppresses the production of farnesyl, an important molecule for the docking of signal proteins to the cell membrane. As a result, apoptosis of carcinogenic bone cells is induced. FPPS is inhibited by bisphosphonates, such as alendronate, pamidronate and zoledronate. For example, the tracer BPAMD with the targeting vector pamidronate is regularly used in the treatment of bone metastases.

A particularly effective tracer for theranostics of bone metastases has been found to be zoledronate (ZOL), a hydroxy bisphosphonate having a heteroaromatic N unit. With the chelators, NODAGA- and DOTA-conjugated zoledronate (scheme 5) are currently the most potent radiotheranostics for bone metastases.

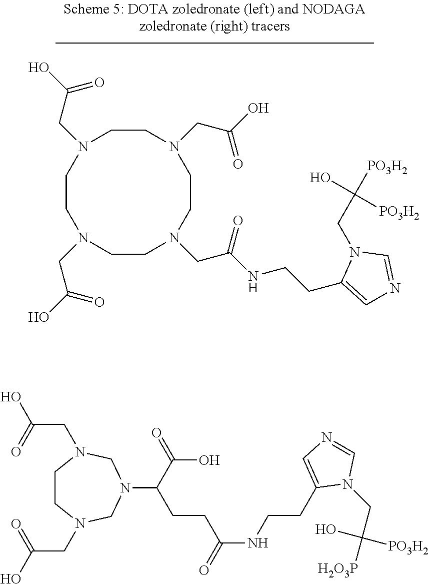

The prior art discloses a multitude of labeling precursors for diagnosis and theranostics of cancers with radioactive isotopes.

WO 2015055318 A1 discloses radiotracers for the diagnosis and theranostics of prostate or epithelial carcinomas, such as, inter alia, the compound PSMA-617 shown in scheme 3.

It is an object of the present invention to provide pharmaceutical compounds and pharmaceutical kits for dual nuclear-medical/cytotoxic theranostics.

This object is achieved by a smart drug delivery system comprising
a first compound having the structure

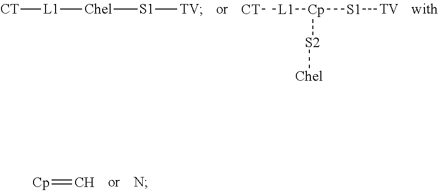

or
a second compound having the structure Chel-S-TV and a third compound having the structure CT-L-TV;

wherein, in the first, second and third compounds,

Chel is a radical of a chelator for the complexation of a radioisotope; CT is a radical of a cytotoxic compound; TV is a biological targeting vector; L1 and L are each a linker; S1, S2 and S are each a spacer.

The invention further provides a pharmaceutical kit for dual nuclear-medical/cytotoxic theranostics, consisting of
a first vessel containing a first compound or a first carrier substance containing the first compound;

or
a second vessel containing a second compound or a second carrier substance containing the second compound, and
a third vessel containing a third compound or a third carrier substance containing the third compound;

wherein

the first compound has the structure

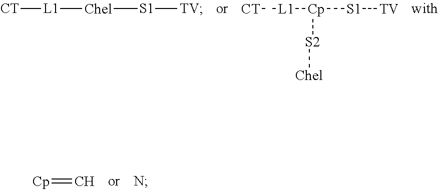

the second compound has the structure Chel-S-TV;

and the third compound has the structure CT-L-TV;

in which

Chel is a radical of a chelator for the complexation of a radioisotope; CT is a radical of a cytotoxic compound; TV is a biological targeting vector; L1 and L are each a linker; S1, S2 and S are each a spacer.

The invention further relates to a compound for dual nuclear-medical/cytotoxic theranostics having the structure

CT-L1-Chel-S1-TV;

in which

Chel is a radical of a chelator for the complexation of a radioisotope; CT is a radical of a cytotoxic compound; TV is a biological targeting vector; L1 is a linker; and S1 is a spacer.

The invention further relates to a compound for dual nuclear-medical/cytotoxic theranostics having the structure

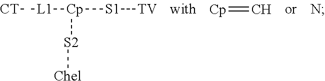

in which

Chel is a radical of a chelator for the complexation of a radioisotope; CT is a radical of a cytotoxic compound; TV is a biological targeting vector; L1 is a linker; S1 and S2 are each a spacer.

Appropriate embodiments of the smart drug delivery system of the invention, of the pharmaceutical kit and of the compounds

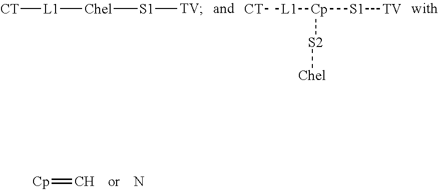

are characterized in that
TV is a targeting vector selected from one of the structures [1] to [18]

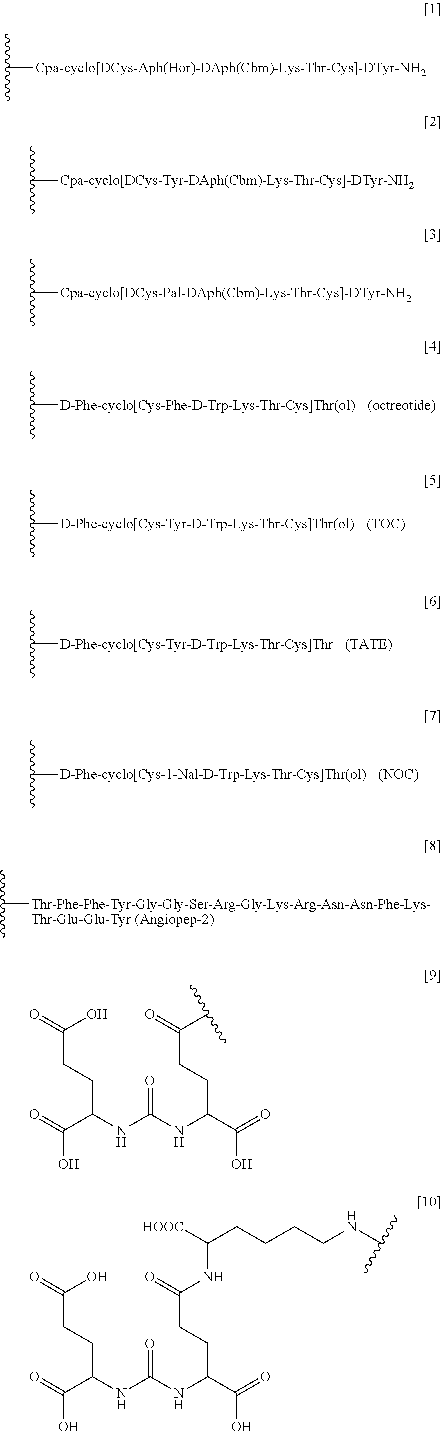

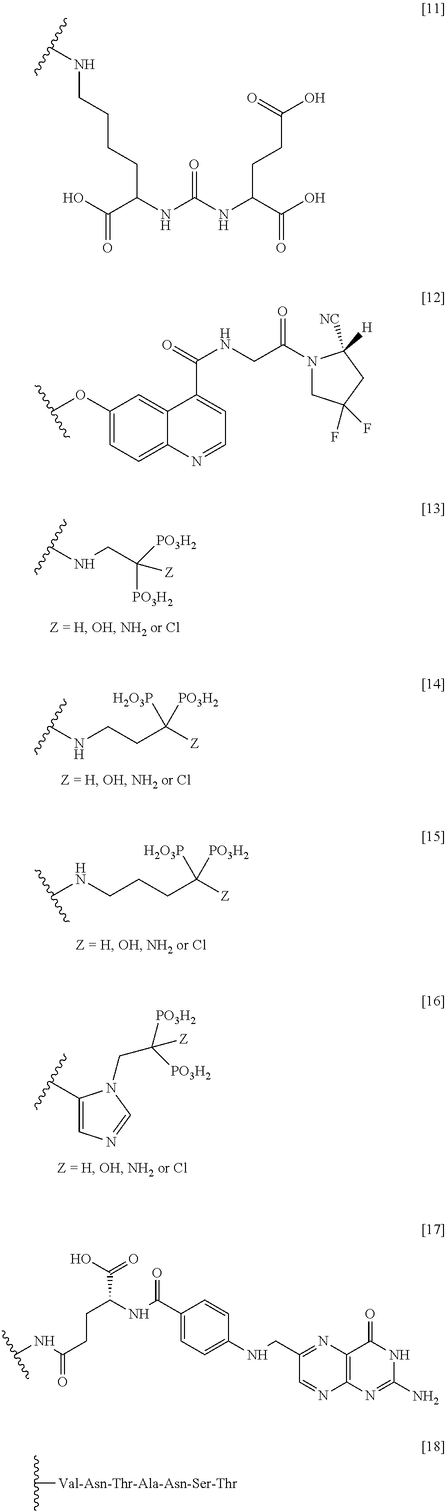

where the structures [1] to [8] and [18] denote amino acid sequences;
L and L1 independently have a structure selected from

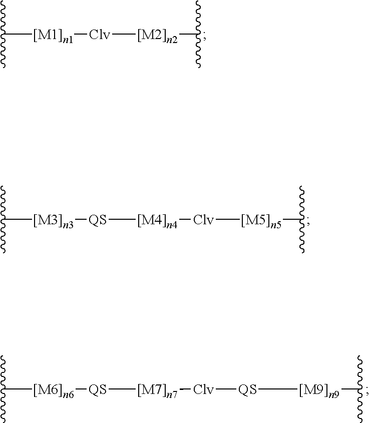

in which M1, M2, M3, M4, M5, M6, M7, M8 and M9 are independently selected from the group comprising amide, carboxamide, phosphinate, alkyl, triazole, thiourea, ethylene, maleimide radicals, —(CH₂)—, —(CH₂CH₂O)—, —CH₂—CH(COOH)—NH— and —(CH₂)ₘNH— with m=1, 2, 3, 4, 5, 6, 7, 8, 9 or 10; and
n1, n2, n3, n4, n5, n6, n7, n8 and n9 are independently selected from the set of {0, 1, 2, 3, 4, 5, 6, 7, 8, 9, 10, 11, 12, 13, 14, 15, 16, 17, 18, 19, 20};
QS is a squaric acid radical

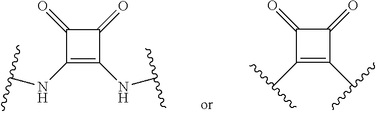 [SMILES: *Nc1c(N*)c(=O)c1=O ; *c1c(*)c(=O)c1=O]

Clv is a cleavable group;
S the same as L (S=L); and/or
S, S1 and S2 independently have a structure selected from

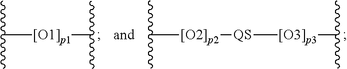

in which O1, O2 and O3 are independently selected from the group comprising amide, carboxamide, phosphinate, alkyl, triazole, thiourea, ethylene, maleimide radicals, —(CH₂)—, —(CH₂CH₂O)—, —CH₂—CH(COOH)—NH— and —(CH₂)_{q}NH— with q=1, 2, 3, 4, 5, 6, 7, 8, 9 or 10; and
p1, p2 and p3 are independently selected from the set of {0, 1, 2, 3, 4, 5, 6, 7, 8, 9, 10, 11, 12, 13, 14, 15, 16, 17, 18, 19, 20};
CT is a radical of a cytotoxic compound selected from adozelesin, alrestatin, anastrozole, anthramycin, bicalutamide, bizelesin, bortezomib, busulfan, camptothecin, capecitabine, carboplatin, carzelesin, CC-1065, chlorambucil, cisplatin, cyclophosphamide, cytarabine (ara-C), dacarbazine (DTIC), dactinomycin, daunorubicin, dexamethasone, disulfiram, docetaxel, doxorubicin, duocarmycin A, duocarmycin B1, duocarmycin B2, duocarmycin C1, duocarmycin C2, duocarmycin D, duocarmycin SA, erismodegib, etoposide (VP-16), fludarabine, fluorouracil (5-FU), flutamide, fulvestrant, gemcitabine, goserelin, idarubicin, ifosfamide, L-asparaginase, leuprolide, lomustine (CCNU), mechlorethamine (nitrogen mustard), megestrol acetatr, melphalan (BCNU), menadione, mertansine, metformin, methotrexate, milataxel, mitoxantrone, monomethylauristatin E (MMAE), motesanib, maytansinoid, napabucasin, NSC668394, NSC95397, paclitaxel, prednisone, pyrrolobenzodiazepine, pyrvinium pamoate, resveratrol, rucaparib, S2, S5, salinomycin, saridegib, shikonin, tamoxifen, temozolomide, tesetaxel, tetrazole, tretinoin, verteporfin, vinblastine, vincristine, vinorelbine, vismodegib, α-chaconine, α-solamargine, α-solanine, α-tomatine;
CT is a radical of a cytotoxic compound selected from the active ingredient groups:
   antimetabolite, such as capecitabine, cytarabine, fludarabine, fluorouracil (5-FU), gemcitabine, methotrexate;
   alkylating cytostatics, such as adozelesin, bizelesin, busulfan, carzelesin, chlorambucil, cyclophosphamide, ifosfamide, lomustine (CCNU), dacarbazine (DTIC), cisplatin, carboplatin, mechlorethamine, melphalan (BCNU), temozolomide;
   topoisomerase inhibitors, such as etoposide (VP-16);
   mitosis inhibitors, such as vinblastine, vincristine, vinorelbine, docetaxel, paclitaxel, tesetaxel, mertansine, milataxel, monomethylauristatin E (MMAE), mytansinoid, napabucasin, saridegib;
   antibiotics, such as dactinomycin, daunorubicin, doxorubicin, duocarmycin A, duocarmycin B1, duocarmycin B2, duocarmycin C1, duocarmycin C2, duocarmycin D, duocarmycin SA, idarubicin anthramycin, salinomycin, mitoxantrone;
   enzyme inhibitors, such as alrestatin, anastrozole, camptothecin, L-asparaginase, motesanib;
   antiandrogens and antiestrogens, such as bicalutamide, flutamide, fulvestrant, tamoxifen, megestrol acetate;
   PARP inhibitors, such as rucaparib, olaparib, niraparib, veliparib, iniparib;
   proteasome inhibitors, such as bortezomib;
   others, such as dexamethasone, disulfiram, erismodegib, goserelin, leuprolide, menadione, metformin, NSC668394, NSC95397, prednisone, pyrrolobenzodiazepine, pyrvinium pamoate, resveratrol, S2, S5, shikonin, tetrazole, tretinoin, verteporfin, vismodegib, α-chaconine, α-solamargine, α-solanine, α-tomatine.
the cleavable group Clv is selected from the group comprising

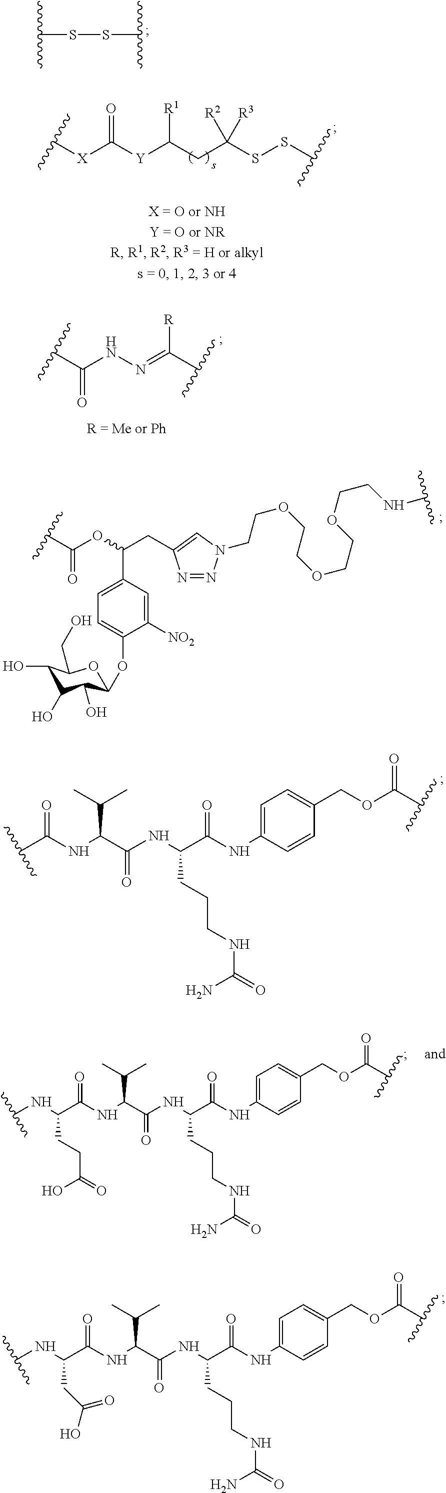

the chelator Chel is selected from the group comprising H₄pypa, EDTA (ethylenediaminetetra-acetate), EDTMP (diethylenetriaminepenta(methylene-phosphonic acid)), DTPA (diethylentriaminepenta-acetate) and derivatives thereof, DOTA (dodeca-1,4,7,10-tetraaminetetraacetate), DOTAGA (2-(1,4,7,10-tetraazacyclododecane-4,7,10)-pentanedioic acid) and other DOTA derivatives, TRITA (trideca-1,4,7,10-tetraaminetetraacetate), TETA (tetradeca-1,4,8,11-tetraaminetetraacetate) and derivatives thereof, NOTA (nona-1,4,7-triamine-triacetate) and derivatives thereof, for example NOTAGA (1,4,7-triazacyclononane,1-glutaric acid,4,7-acetate), TRAP (triazacyclononanephosphinic acid), NOPO (1,4,7-triazacyclononane-1,4-bis[methylene(hydroxymethyl)phosphinic acid]-7-[methylene(2-carboxyethyl) phosphinic acid]), PEPA (pentadeca-1,4,7,10,13-pentaaminepentaacetate), HEHA (hexadeca-1,4,7,10,13,16-hexaaminehexaacetate) and derivatives thereof, HBED (hydroxybenzylethylene-diamine) and derivatives thereof, DEDPA and derivatives thereof, such as H₂DEDPA (1,2-[[6-(carboxylate-)pyridin-2-yl]methylamino]ethane), DFO (deferoxamine) and derivatives thereof, trishydroxypyridinone (THP) and derivatives thereof, such as YM103, TEAP (tetraazycyclodecanephosphinic acid) and derivatives thereof, AAZTA (6-amino-6-methylperhydro-1,4-diazepine-N,N,N′,N′-tetraacetate) and derivatives, such as DATA ((6-pentanoic acid)-6-(amino)methyl-1,4-diazepine triacetate); SarAr (1-N-(4-aminobenzyl)-3,6,10,13,16,19-hexaazabicyclo[6.6.6]-eicosane-1,8-diamine) and salts thereof, (NH₂)₂SAR (1,8-diamino-3,6,10,13,16,19-hexaazabicyclo[6.6.6]eicosane) and salts and derivatives thereof, aminothiols and derivatives thereof; and/or
the first, second and third carrier substance are independently selected from the group comprising water, 0.45% aqueous NaCl solution, 0.9% aqueous NaCl solution, Ringer's solution (Ringer's lactate), 5% aqueous dextrose solution and aqueous alcohol solutions.

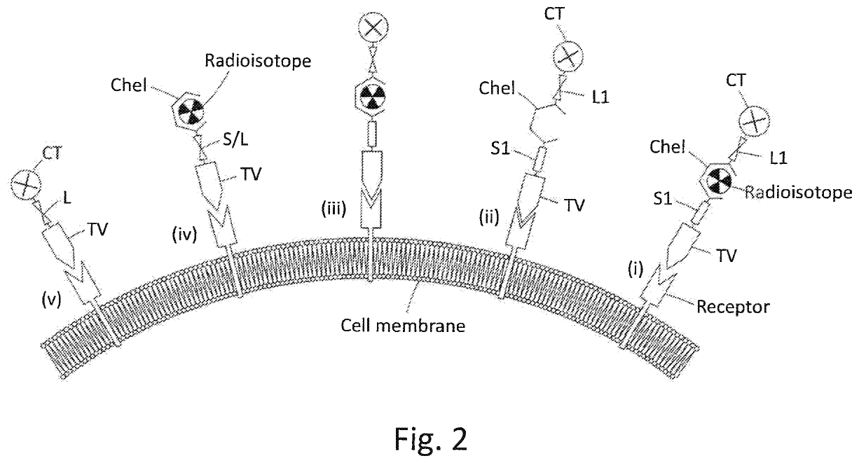

The smart drug delivery system and pharmaceutical kit of the invention enable a new form of targeted dual cancer treatment with a diagnostic and therapeutic modality (see FIG. 2 and table 1). This involves using the same active ingredient conjugate or two biologically and pharmacokinetically analogous active ingredient conjugates in low and elevated dose.

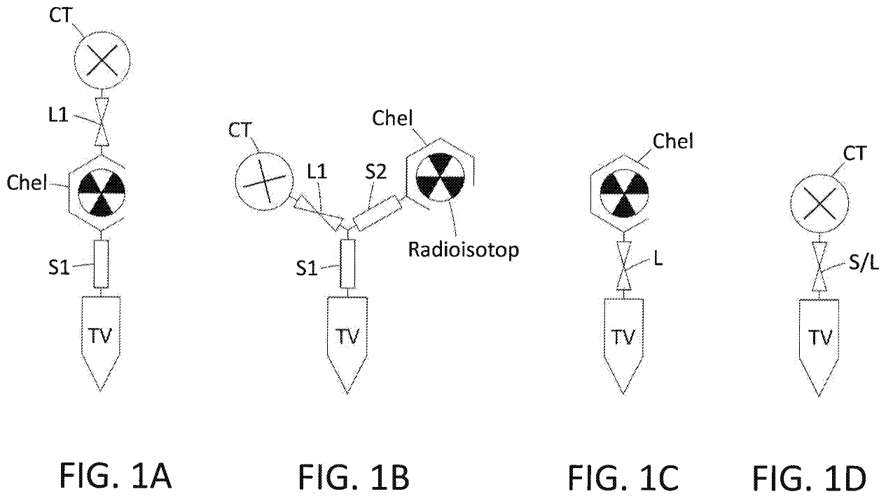

The structure of compounds or active ingredient conjugates of the invention is shown schematically in FIGS. 1a to 1d, where CT denotes a cytotoxic group; L, L1 each denote a cleavable linker group; Chel denotes a chelator for labeling with a radioisotope; S denotes a cleavable linker or spacer group; S1, S2 each denote a spacer group and TV denotes a biological targeting vector.

The diagnostic and therapeutic modalities provided by the invention are illustrated in FIG. 2 by five membrane-bound receptors (i) to (v), where the designations CT, L, L1, Chel, S, S1, S2 and TV have the same meaning as elucidated above in connection with FIGS. 1a to 1d. The receptors (i)-(v) shown in FIG. 2, in table 1, are assigned the diagnostic and therapeutic modalities (A), (B1), (B2) and (C), (D1), (D2), each in conjunction with a qualitative dose indication.

TABLE 1
Diagnostic and therapeutic modalities according to FIG. 1

| Receptor | Modality | | Dose |
|---|---|---|---|
| (i) | (A) | Nuclear-medical diagnosis | low |
| (ii) | (B1) | Cytotoxic treatment | elevated |
| (iii) | (B2) | Nuclear-medical/cytotoxic treatment | elevated |
| (iv) | (C) | Nuclear-medical diagnosis | low |
| (v) | (D1) | Cytotoxic treatment | elevated |
| (iv) + (v) | (D2) | Nuclear-medical/cytotoxic treatment | elevated |

In the case of the modalities (A), (B1), (B2) listed in table 1, the same active ingredient conjugate is used with radioisotope (A, B2) and without radioisotope (B1). A cancer cell, after endocytosis and cleavage of the linker L1 in the case of modality (B1), is subjected merely to the cytotoxic active ingredient CT, and in the case of modality (B2) to the cytotoxic active ingredient CT and simultaneously to the radiation emitted by the radioisotope.

In the case of modality (D2), two analogous active ingredient conjugates are utilized with radioisotope (iv) and without radioisotope (v).

The targeting vectors TV used in accordance with the invention have a high binding affinity to a membrane-bound receptor. The receptors addressed in the present invention are proteins, for example prostate-specific membrane antigen (PSMA), fibroblast activation protein (FAP) or farnesyl pyrophosphate synthase (FPPS), which are overexpressed on the envelope of tumor cells in various cancers.

The spacers S, S1, S2 bind the chelator Chel to the targeting vector TV and at the same time function as spacer and chemical modulator that compensates for any impairment of binding affinity of the targeting vector TV caused by the chelator Chel, for example owing to steric hindrance.

In an analogous manner, the linkers L and L1, and any spacer S identical to L, bind the chelator Chel to the cytotoxic active ingredient CT or to the targeting vector TV and modulate the pharmacokinetic properties. Numerous cytotoxic active ingredients are hydrophobic and sparingly soluble in the blood serum. Significant lipophobicity of a cytotoxic active ingredient CT can be effectively compensated for, inter alia, with the aid of a polyethylene glycol (PEG)-containing linker L, L1. This approach is known in the state of the art by the name “PEGylation”.

The linkers L and L1 further include a group Clv which, after uptake into a tumor cell (endocytosis), is cleaved by enzymes or molecules present in late endosomes or in lysosomes, for example glutathione (γ-L-glutamyl-L-cysteinylglycine, abbreviated to GSH), and releases the cytotoxic active ingredient CT.

The linkers L, L1 are crucial to the pharmacokinetic properties and embody a central starting point for the invention which is based on one identical or two biologically analogous active ingredient conjugates for dual nuclear-medical and cytotoxic treatment, and enables direct translation from diagnosis to treatment.

The present invention further provides a pharmaceutical kit for targeted, simultaneous nuclear-medical/cytotoxic cancer treatment according to the above-elucidated modalities (B2) and (D2). First, using a radioisotope suitable for molecular imaging by means of PET or SPECT, it is ascertained whether the targeting vector of the smart drug delivery system binds to a molecular target which is expressed in sufficient quantity by the patient's tumor tissue. For example, a smart drug delivery system with a PSMA inhibitor as targeting vector is used in patients with prostrate carcinoma, and must show sufficiently high and selective accumulation in the primary tumor, in metastases of the lymph system, the viscera or bones. In this case, the smart drug delivery system (SDDS) serves as a pre-therapeutic diagnostic agent and indicates the suitability of the treatment for the respective patient. Since the same SDDS is involved, identical pharmacokinetic and pharmacodynamic properties are assured. The patient's response level can be predicted here with high certainty. Known SDDSs contain merely a cytostatic coupled to a targeting vector. Therefore, in the case of use of known SDDSs, suitability for the patient is not ascertained before commencement of treatment. At most the patient's target expression is determined by means of a PET radiotracer other than the SDDS. However, the PET signal measured by means of a separate PET tracers is not representative of the binding and pharmacokinetics of the SDDS. But the latter is crucial for the efficacy and the penetration of systemic barriers, and for the judgement of dose. This is particularly true of metastasizing prostate carcinomas, were 11.8% of patients affected have a mutation in DNA repair genes (cf. C. C. Pritchard, J. Mateo, M. F. Walsh, N. De Sarkar, W. Abida, H. Beltran, A. Garofalo, R. Gulati, S. Carreira, R. Eeles, O. Elemento, M. A. Rubin et al.; Inherited DNA-Repair Gene Mutations in Men with Metastatic Prostate Cancer; N Engl J Med 2016; 375:443-453; doi: 10.1056/NEJMoa1603144; C. Kratochwil, F. L. Giesel, C.-P. Heussel, D. Kazdal, V. Endris, C. Nientiedt, F. Bruchertseifer, M. Kippenberger, H. Rathke, J. Leichsenring, M. Hohenfellner, A. Morgenstern, U. Haberkorn, S. Duensing, and A. Stenzinger; Patients Resistant Against PSMA-Targeting α-Radiation Therapy Often Harbor Mutations in DNA Damage-Repair-Associated Genes; doi: 10.2967/jnumed.119.234559). The compounds of the invention determine at the pre-treatment stage whether the treatment is suitable for the patient, both in relation to target expression and the pharmacokinetic profile. In conjunction with radiosensitizing PARPi, such as the above-described rucaparib in particular, an effective therapeutic approach is established. There are numerous studies in respect of use of rucaparib in combination with radiotherapy.

According to the indication, the treatment can be effected without or with radiolabeling of the smart drug delivery system, i.e. by purely cytotoxic or nuclear-medical/cytotoxic means. In the latter case, on account of the locally high radiation dose, reactive free radicals (reactive oxygen species: ROS) are formed, and ABC transporter channels (ATP binding cassette: ABC), for example P-gp or Ptch1, that are crucial to the resistance (multidrug resistance: MDR) of cancer cells are inactivated, and the discharge (exocytosis) of the cytotoxic compound CT from the cancer cell is inhibited.

Cytotoxic Compound CT (Cytostatics)

The state of the art discloses a multitude of cytotoxic active ingredients for cancer treatment.

For example, rucaparib and some of its derivatives inhibit the enzyme PARP (poly-ADP-ribose polymerase), which is involved in the repair of single-strand breaks (SSBs) in DNA. The effect of PARP inhibitors is based on synthetically induced lethality. In a healthy cell with DNA in the intact state, PARP inhibition does not lead to cell death because double-strand breaks (DSBs) in the DNA that have resulted from SSBs are repaired by homologous recombination (HR). In HR-deficient cells, PARP inhibition, by contrast, leads to cell death since DSBs accumulate in the cell and recruit apoptosis molecules. The two genes BRCA1 and BRCA2 (breast cancer genes) are crucially involved in HR. A mutation in these genes leads to disruption of the DNA repair and increases the risk of tumor formation.

In 20-25% of patients with mCRPC (metastasized castration-resistant prostate carcinoma), HR genes, including BRCA1/2, are mutated. These patients benefit from a treatment with PARP inhibitors having high tumor specificity. It is also possible to pharmaceutically induce BRCA deficiency. The active ingredient enzalutamide, an inhibitor of the androgen receptor signaling pathway, can result in down-regulation of the BRCA genes. After administration of enzalutamide, even patients without a BRCA mutation can benefit from the selective tumor toxicity of rucaparib. The patient collective for PARP treatment can thus be extended.

Docetaxel and paclitaxel belong to the group of taxanes. Taxanes inhibit the depolymerization of microtubuli and inhibit mitosis (cell division).

Temozolomide is a pharmaceutically adapted active ingredient (prodrug), which, after metabolization and spontaneous hydrolytic cleavage, releases methylhydrazine (CH₃(NH)NH₂), which methylates DNA basis and induces apoptosis.

Monomethyl-auristatin E (MMAE) is an antineoplastic active ingredient that interrupts the cell cycle by inhibition of tubulin polymerization and hence leads to apoptosis.

Table 2 shows cytostatics used in accordance with the invention.

TABLE 2
Active cytostatic ingredients (CTs) used in accordance with the invention

| | |
|---|---|
| Adozelesin | 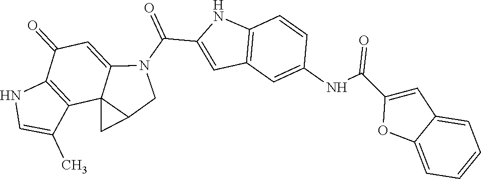 [SMILES: Cc1c[nH]c2c1C13CC1CN(C(=O)c1cc4cc(NC(=O)c5cc6ccccc6o5)ccc4[nH]1)C3=CC2=O] |
| Alrestatin | 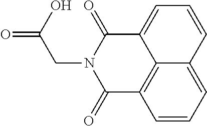 [SMILES: O=C(O)CN1C(=O)c2cccc3cccc(c23)C1=O] |
| Anastrozole | 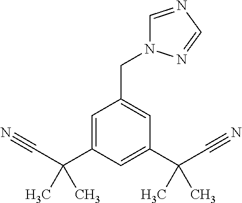 [SMILES: CC(C)(C#N)c1cc(Cn2cncn2)cc(C(C)(C)C#N)c1] |
| Anthramycin | 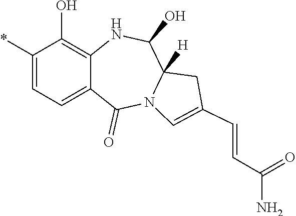 [SMILES: *c1ccc2c(c1O)N[C@@H](O)[C@@H]1CC(/C=C/C(N)=O)=CN1C2=O] |
| Bicalutamide | 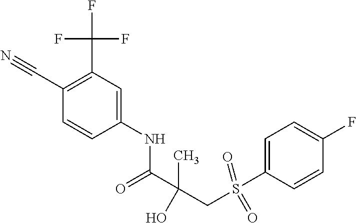 [SMILES: CC(O)(CS(=O)(=O)c1ccc(F)cc1)C(=O)Nc1ccc(C#N)c(C(F)(F)F)c1] |
| Bizelesin | 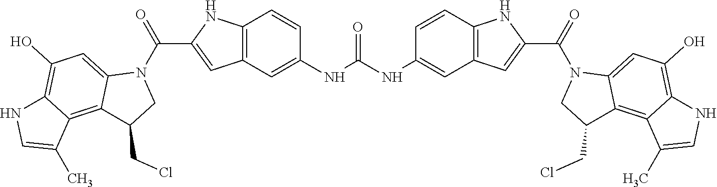 [SMILES: Cc1c[nH]c2c(O)cc3c(c12)[C@H](CCl)CN3C(=O)c1cc2cc(NC(=O)Nc3ccc4[nH]c(C(=O)N5C[C@@H](CCl)c6c5cc(O)c5[nH]cc(C)c65)cc4c3)ccc2[nH]1] |
| Bortezomib | 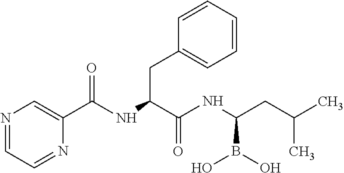 [SMILES: CC(C)C[C@H](NC(=O)[C@H](Cc1ccccc1)NC(=O)c1cnccn1)B(O)O] |
| Busulfan | 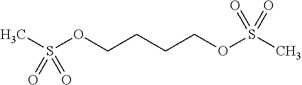 |
| Camptothecin | 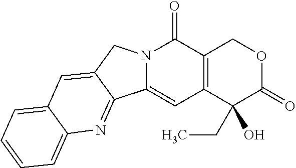 [SMILES: CC[C@@]1(O)C(=O)OCc2c1cc1n(c2=O)Cc2cc3ccccc3nc2-1] |
| Capecitabine | 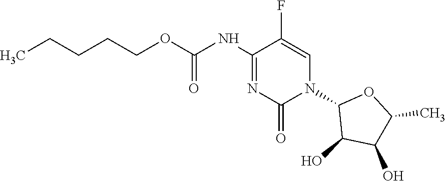 [SMILES: CCCCCOC(=O)Nc1nc(=O)n([C@@H]2O[C@H](C)[C@@H](O)[C@H]2O)cc1F] |
| Carboplatin | 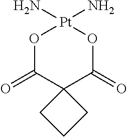 |
| Carzelesin | 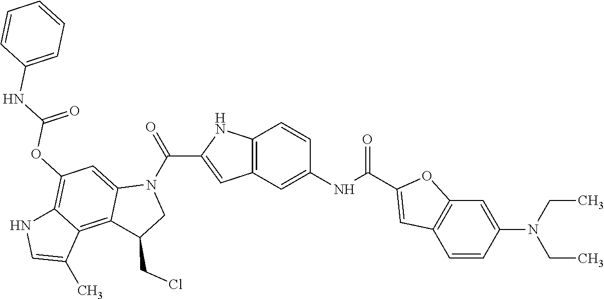 [SMILES: CCN(CC)c1ccc2cc(C(=O)Nc3ccc4[nH]c(C(=O)N5C[C@@H](CCl)c6c5cc(OC(=O)Nc5ccccc5)c5[nH]cc(C)c65)cc4c3)oc2c1] |
| CC-1065 | 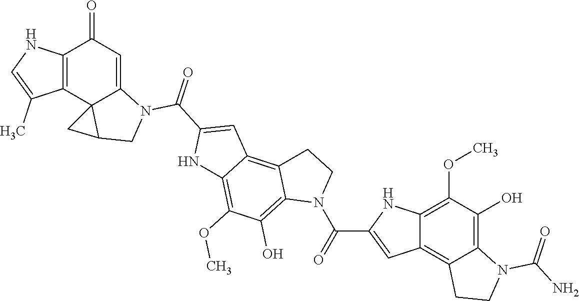 [SMILES: COc1c(O)c2c(c3cc(C(=O)N4CCc5c4c(O)c(OC)c4[nH]c(C(=O)N6CC7CC78C6=CC(=O)c6[nH]cc(C)c68)cc54)[nH]c13)CCN2C(N)=O] |
| Chlorambucil | 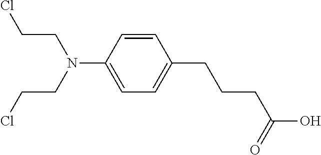 [SMILES: O=C(O)CCCc1ccc(N(CCCl)CCCl)cc1] |
| Cisplatin | 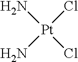 |
| Cyclophosphamide | 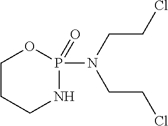 [SMILES: O=P1(N(CCCl)CCCl)NCCCO1] |
| Cytarabine (ara-C) | 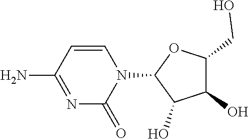 [SMILES: Nc1ccn([C@@H]2O[C@H](CO)[C@@H](O)[C@@H]2O)c(=O)n1] |
| Dacarbazine (DTIC) | 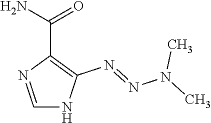 |
| Dactinomycin | 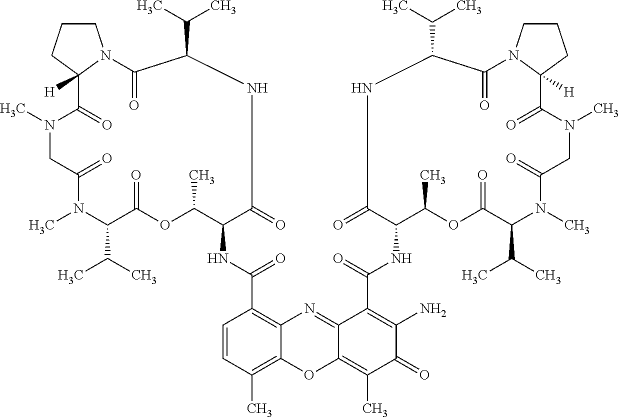 |
| Daunorubicin | 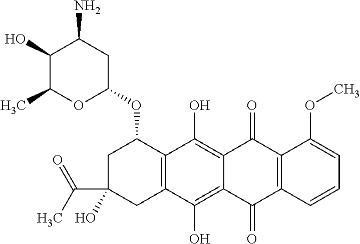 [SMILES: COc1cccc2c1C(=O)c1c(O)c3c(c(O)c1C2=O)C[C@@](O)(C(C)=O)C[C@@H]3O[C@H]1C[C@H](N)[C@H](O)[C@H](C)O1] |
| Dexamethasone | 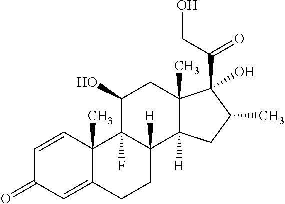 |
| Disulfiram | 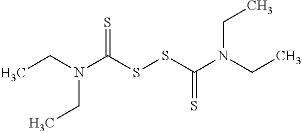 [SMILES: CCN(CC)C(=S)SSC(=S)N(CC)CC] |
| Docetaxel | 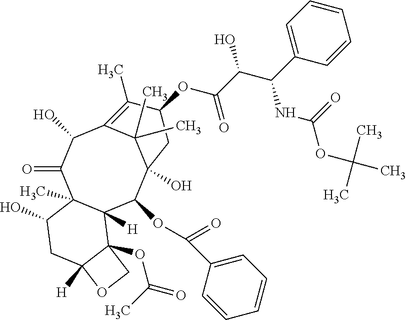 [SMILES: CC(=O)O[C@@]12CO[C@@H]1C[C@H](O)[C@@]1(C)C(=O)[C@H](O)C3=C(C)[C@@H](OC(=O)[C@H](O)[C@@H](NC(=O)OC(C)(C)C)c4ccccc4)C[C@@](O)([C@@H](OC(=O)c4ccccc4)[C@H]21)C3(C)C] |
| Doxorubicin | 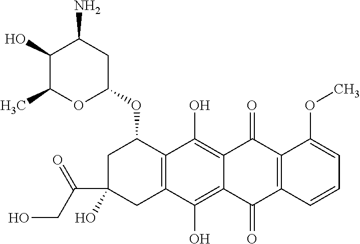 [SMILES: COc1cccc2c1C(=O)c1c(O)c3c(c(O)c1C2=O)C[C@@](O)(C(=O)CO)C[C@@H]3O[C@H]1C[C@H](N)[C@H](O)[C@H](C)O1] |
| Duocarmycin A | 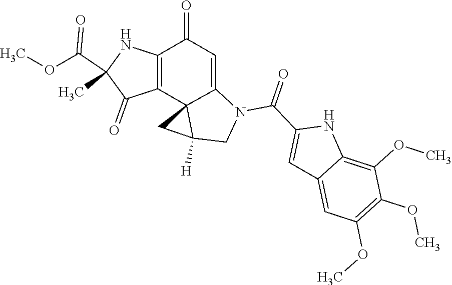 |
| Duocarmycin B1 | 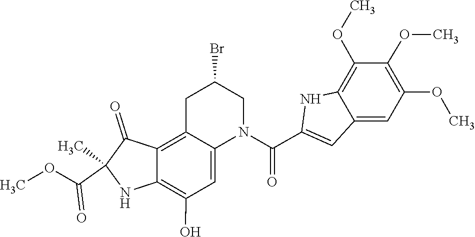 |
| Duocarmycin B2 | 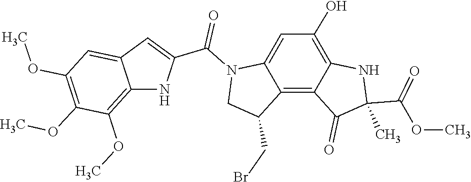 |
| Duocarmycin C1 | 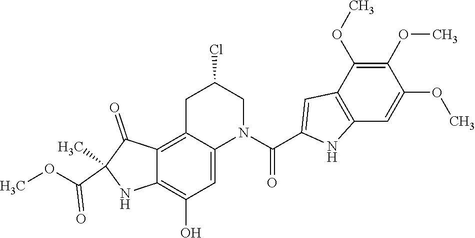 |
| Duocarmycin C2 | 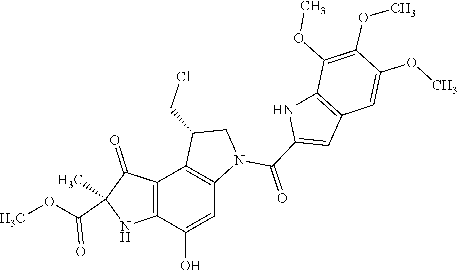 |
| Duocarmycin D | 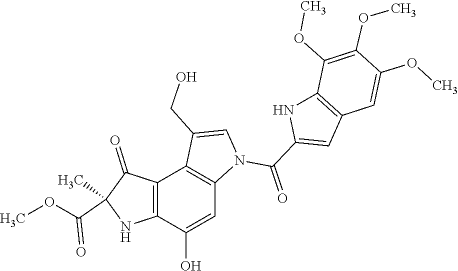 |
| Duocarmycin SA | 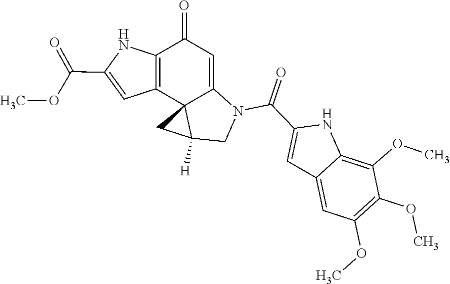 |
| Erismodegib | 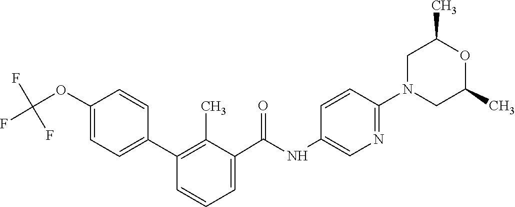 [SMILES: Cc1c(C(=O)Nc2ccc(N3C[C@@H](C)O[C@@H](C)C3)nc2)cccc1-c1ccc(OC(F)(F)F)cc1] |
| Etoposide (VP-16) | 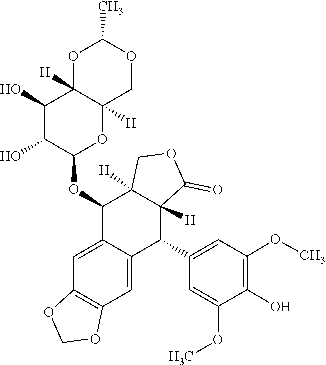 [SMILES: COc1cc([C@@H]2c3cc4c(cc3[C@@H](O[C@@H]3O[C@@H]5CO[C@@H](C)O[C@H]5[C@H](O)[C@H]3O)[C@H]3COC(=O)[C@H]23)OCO4)cc(OC)c1O] |
| Fludarabine | 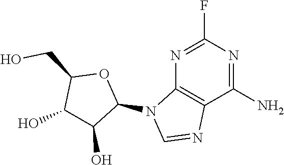 [SMILES: Nc1nc(F)nc2c1ncn2[C@@H]1O[C@H](CO)[C@@H](O)[C@@H]1O] |
| Fluorouracil (5-FU) | 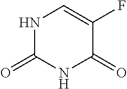 |
| Flutamide | 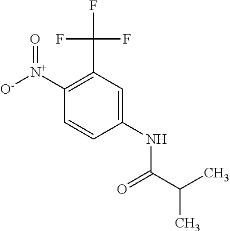 [SMILES: CC(C)C(=O)Nc1ccc([N+](=O)[O-])c(C(F)(F)F)c1] |
| Fulvestrant | 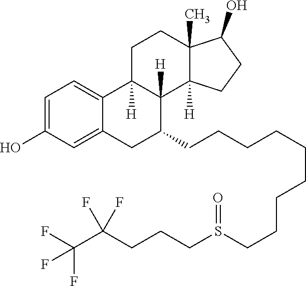 |
| Gemcitabine | 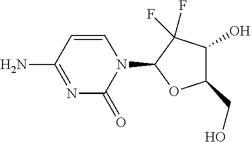 [SMILES: Nc1ccn([C@@H]2O[C@H](CO)[C@@H](O)C2(F)F)c(=O)n1] |
| Goserelin | 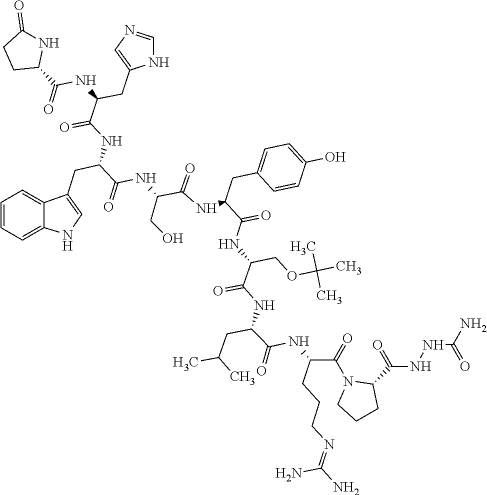 [SMILES: CC(C)C[C@H](NC(=O)[C@@H](COC(C)(C)C)NC(=O)[C@H](Cc1ccc(O)cc1)NC(=O)[C@H](CO)NC(=O)[C@H](Cc1c[nH]c2ccccc12)NC(=O)[C@H](Cc1cnc[nH]1)NC(=O)[C@@H]1CCC(=O)N1)C(=O)N[C@@H](CCCN=C(N)N)C(=O)N1CCC[C@H]1C(=O)NNC(N)=O] |
| Idarubicin | 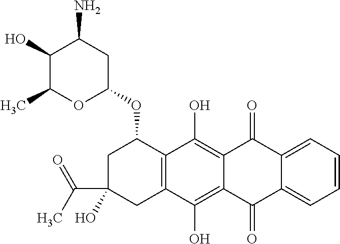 [SMILES: CC(=O)[C@]1(O)Cc2c(O)c3c(c(O)c2[C@@H](O[C@H]2C[C@H](N)[C@H](O)[C@H](C)O2)C1)C(=O)c1ccccc1C3=O] |
| Ifosfamide | 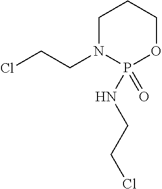 [SMILES: O=P1(NCCCl)OCCCN1CCCl] |
| L-Asparaginase* | MEFFKKTALAALVMGFSGAALALPNITILATGGTIA |
| | GGGDSATKSNYTVGKVGVENLVNA |
| | VPQLKDIANVKGEQVVNIGSQDMNDNVWLTLAK |
| | KINTDCDKTDGFVITHGTDTMEETAYF |
| | LDLTVKCDKPVVMVGAMRPSTSMSADGPFNLYNA |
| | VVTAADKASANRGVLVVMNDTVLDGR |
| | DVTKTNTTDVATFKSVNYGPLGYIHNGKIDYQRTP |
| | ARKHTSDTPFDVSKLNELPKVGIVY |
| | NYANASDLPAKALVDAGYDGIVSAGVGNGNLYKS |
| | VFDTLATAAKTGTAVVRSSRVPTGAT |
| | TQDAEVDDAKYGFVASGTLNPQKARVLLQLALTQ |
| | TKDPQQIQQIFNQY |
| Leuprolide* | PHWSYLLR |
| Lomustine (CCNU) | 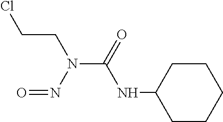 [SMILES: O=NN(CCCl)C(=O)NC1CCCCC1] |
| Mechlorethamine (nitrogen mustard) | 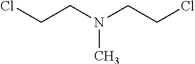 |
| Megestrol acetate | 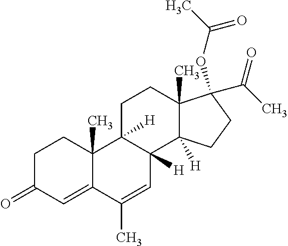 |
| Melphalan (BCNU) | 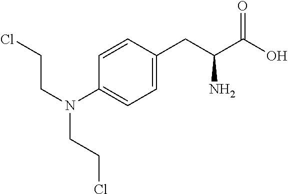 [SMILES: N[C@@H](Cc1ccc(N(CCCl)CCCl)cc1)C(=O)O] |
| Menadione | 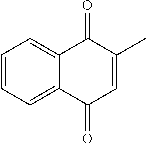 |
| Mertansine | 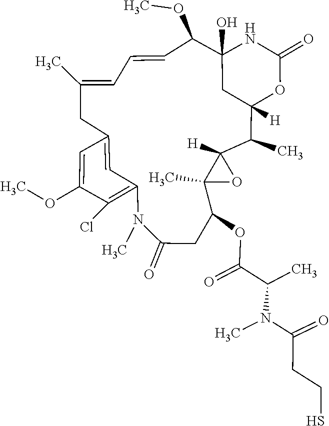 [SMILES: COc1cc2cc(c1Cl)N(C)C(=O)C[C@H](OC(=O)[C@H](C)N(C)C(=O)CCS)[C@]1(C)O[C@H]1[C@H](C)[C@@H]1C[C@@](O)(NC(=O)O1)[C@H](OC)/C=C/C=C(\C)C2] |
| Metformin | 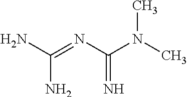 |
| Methotrexate | 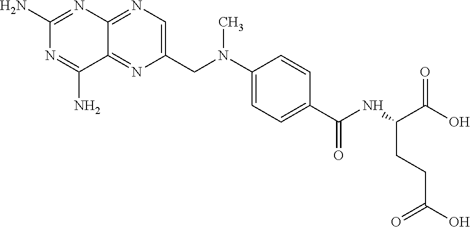 [SMILES: CN(Cc1cnc2nc(N)nc(N)c2n1)c1ccc(C(=O)N[C@@H](CCC(=O)O)C(=O)O)cc1] |
| Milataxel | 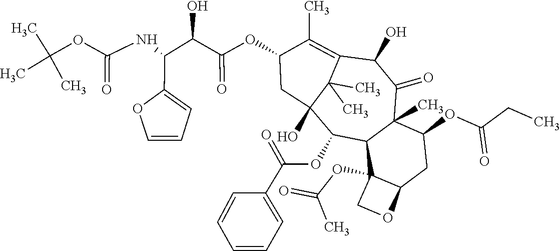 [SMILES: CCC(=O)O[C@H]1C[C@H]2OC[C@@]2(OC(C)=O)[C@H]2[C@H](OC(=O)c3ccccc3)[C@]3(O)C[C@H](OC(=O)[C@H](O)[C@@H](NC(=O)OC(C)(C)C)c4ccco4)C(C)=C([C@@H](O)C(=O)[C@]12C)C3(C)C] |
| Mitoxantrone | 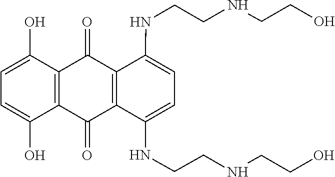 [SMILES: O=C1c2c(O)ccc(O)c2C(=O)c2c(NCCNCCO)ccc(NCCNCCO)c21] |
| Monomethyl-auristatin E (MMAE) | 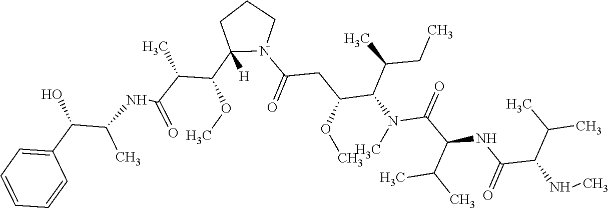 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@@H](NC)C(C)C)C(C)C] |
| Motesanib | 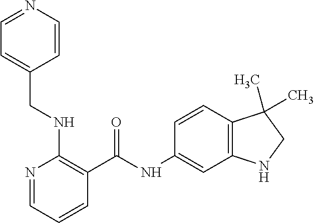 [SMILES: CC1(C)CNc2cc(NC(=O)c3cccnc3NCc3ccncc3)ccc21] |
| Mytansinoid | 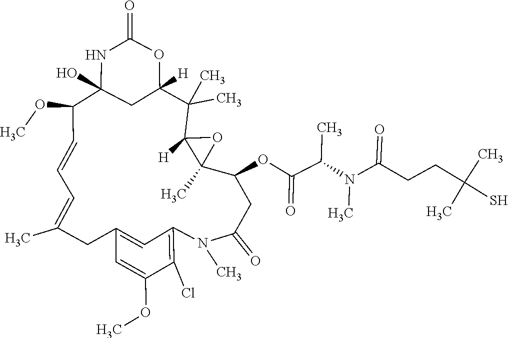 [SMILES: COc1cc2cc(c1Cl)N(C)C(=O)C[C@H](OC(=O)[C@H](C)N(C)C(=O)CCC(C)(C)S)[C@]1(C)O[C@H]1C(C)(C)[C@@H]1C[C@@](O)(NC(=O)O1)[C@H](OC)/C=C/C=C(\C)C2] |
| Napabucasin | 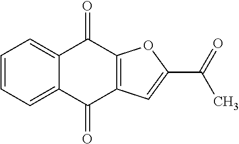 [SMILES: CC(=O)c1cc2c(o1)C(=O)c1ccccc1C2=O] |
| NSC668394 | 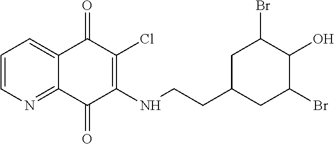 [SMILES: O=C1C(Cl)=C(NCCC2CC(Br)C(O)C(Br)C2)C(=O)c2ncccc21] |
| NSC95397 | 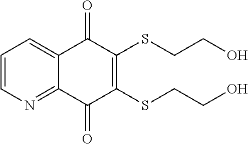 [SMILES: O=C1C(SCCO)=C(SCCO)C(=O)c2ncccc21] |
| Paclitaxel | 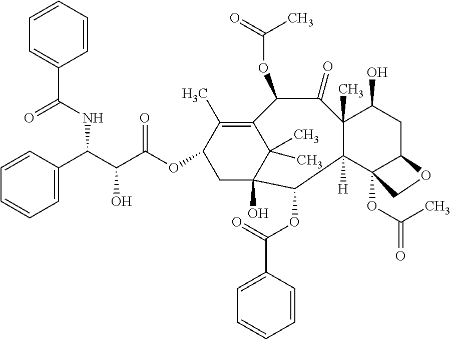 [SMILES: CC(=O)O[C@H]1C(=O)[C@@]2(C)[C@H]([C@H](OC(=O)c3ccccc3)[C@]3(O)C[C@H](OC(=O)[C@H](O)[C@@H](NC(=O)c4ccccc4)c4ccccc4)C(C)=C1C3(C)C)[C@]1(OC(C)=O)CO[C@@H]1C[C@@H]2O] |
| Prednisone | 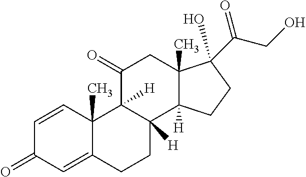 |
| Pyrrolobenzo-diazepine | 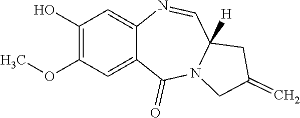 |
| Pyrvinium pamoate | 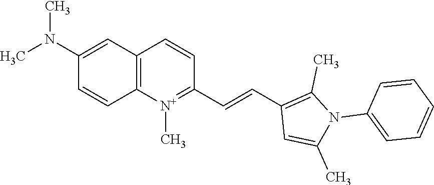 [SMILES: Cc1cc(/C=C/c2ccc3cc(N(C)C)ccc3[n+]2C)c(C)n1-c1ccccc1] |
| | 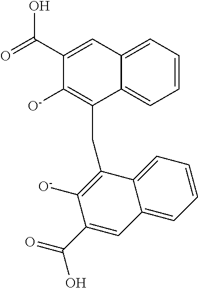 [SMILES: O=C(O)c1cc2ccccc2c(Cc2c([O-])c(C(=O)O)cc3ccccc23)c1[O-]] |
| Resveratrol | 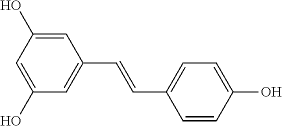 [SMILES: Oc1ccc(/C=C/c2cc(O)cc(O)c2)cc1] |
| Rucaparib | 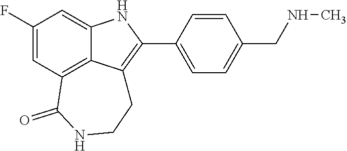 [SMILES: CNCc1ccc(-c2[nH]c3cc(F)cc4c3c2CCNC4=O)cc1] |
| S2 | 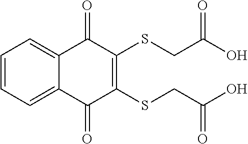 [SMILES: O=C(O)CSC1=C(SCC(=O)O)C(=O)c2ccccc2C1=O] |
| S5 | 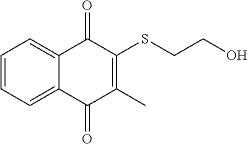 [SMILES: CC1=C(SCCO)C(=O)c2ccccc2C1=O] |
| Salinomycin | 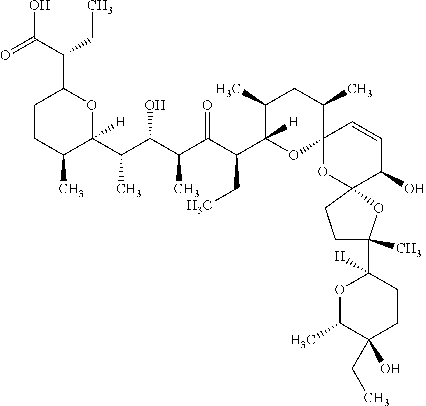 |
| Saridegib | 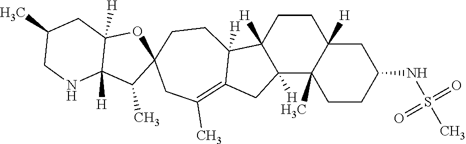 [SMILES: CC1=C2C[C@H]3[C@@H](CC[C@@H]4C[C@H](NS(C)(=O)=O)CC[C@@]43C)[C@@H]2CC[C@@]2(C1)O[C@@H]1C[C@H](C)CN[C@H]1[C@H]2C] |
| Shikonin | 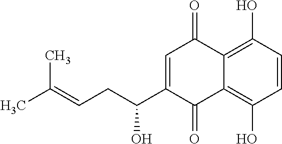 [SMILES: CC(C)=CC[C@@H](O)C1=CC(=O)c2c(O)ccc(O)c2C1=O] |
| Tamoxifen | 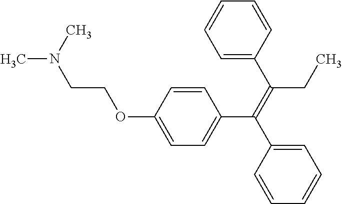 [SMILES: CC/C(=C(\c1ccccc1)c1ccc(OCCN(C)C)cc1)c1ccccc1] |
| Temozolomide | 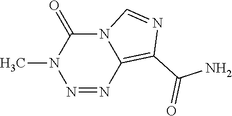 |
| Tesetaxel | 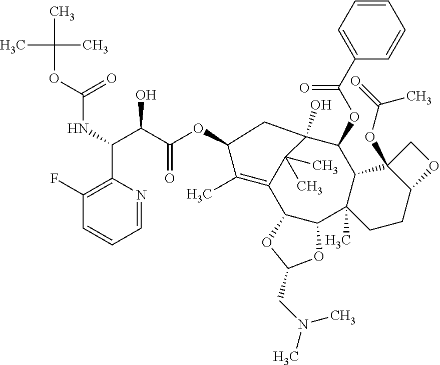 [SMILES: CC(=O)O[C@@]12CO[C@@H]1CC[C@@]1(C)[C@@H]3O[C@H](CN(C)C)O[C@@H]3C3=C(C)[C@@H](OC(=O)[C@H](O)[C@@H](NC(=O)OC(C)(C)C)c4ncccc4F)C[C@@](O)([C@@H](OC(=O)c4ccccc4)[C@@H]12)C3(C)C] |
| Tetrazole | 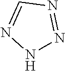 |
| Tretinoin | 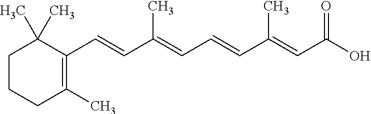 [SMILES: CC1=C(/C=C/C(C)=C/C=C/C(C)=C/C(=O)O)C(C)(C)CCC1] |
| Verteporfin | 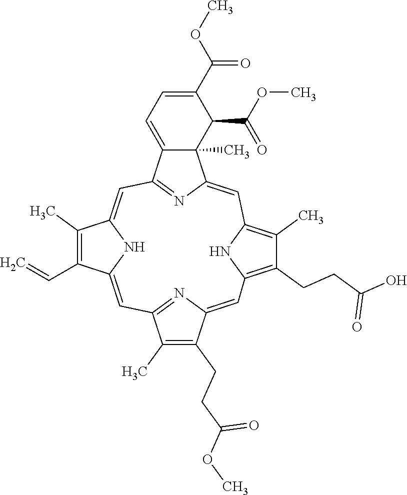 [SMILES: C=Cc1c(C)c2cc3nc(cc4[nH]c(cc5nc(cc1[nH]2)C(C)=C5CCC(=O)OC)c(CCC(=O)O)c4C)[C@@]1(C)C3=CC=C(C(=O)OC)[C@H]1C(=O)OC] |
| | 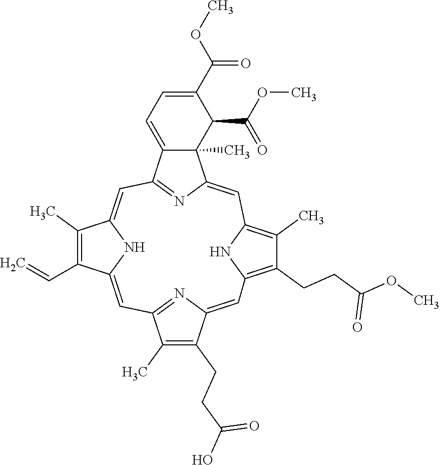 [SMILES: C=Cc1c(C)c2cc3nc(cc4[nH]c(cc5nc(cc1[nH]2)C(C)=C5CCC(=O)O)c(CCC(=O)OC)c4C)[C@@]1(C)C3=CC=C(C(=O)OC)[C@H]1C(=O)OC] |
| Vinblastine | 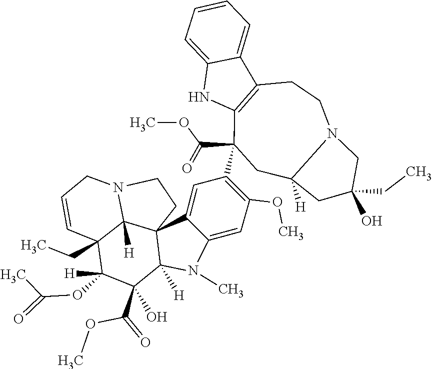 |
| Vincristine | 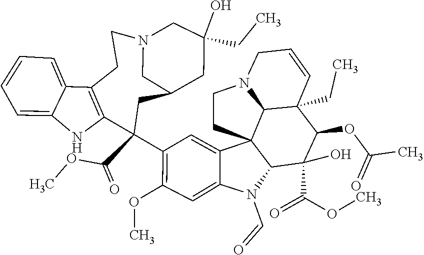 [SMILES: CC[C@]1(O)C[C@@H]2CN(CCc3c([nH]c4ccccc34)[C@@](C(=O)OC)(c3cc4c(cc3OC)N(C=O)[C@H]3[C@@](O)(C(=O)OC)[C@H](OC(C)=O)[C@]5(CC)C=CCN6CC[C@]43[C@@H]65)C2)C1] |
| Vinorelbine | 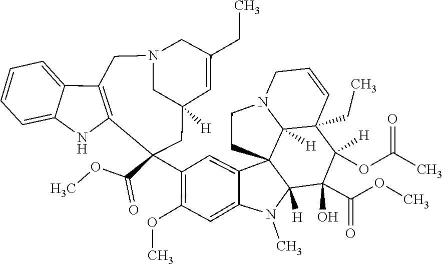 |
| Vismodegib | 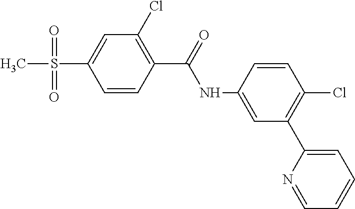 [SMILES: CS(=O)(=O)c1ccc(C(=O)Nc2ccc(Cl)c(-c3ccccn3)c2)c(Cl)c1] |
| α-Chaconine | 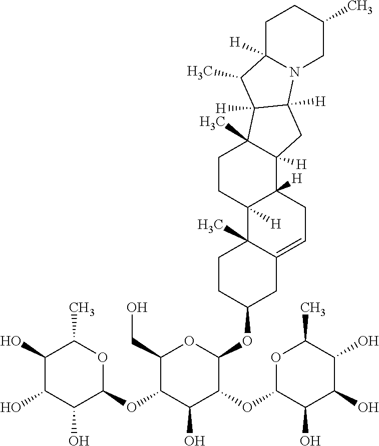 |
| α-Solamargine | 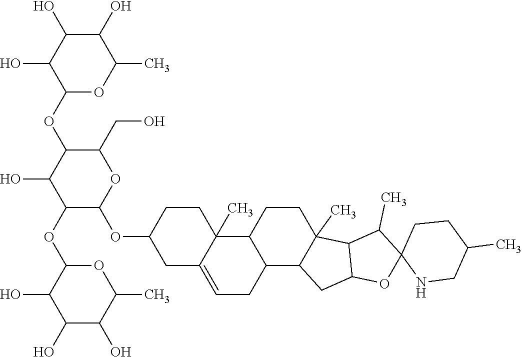 [SMILES: CC1CCC2(NC1)OC1CC3C4CC=C5CC(OC6OC(CO)C(OC7OC(C)C(O)C(O)C7O)C(O)C6OC6OC(C)C(O)C(O)C6O)CCC5(C)C4CCC3(C)C1C2C] |
| α-Solanine | 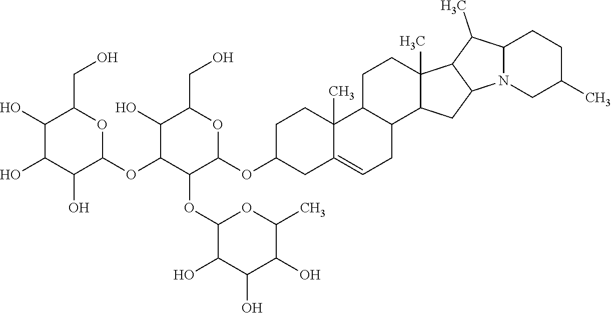 [SMILES: CC1CCC2C(C)C3C(CC4C5CC=C6CC(OC7OC(CO)C(O)C(OC8OC(CO)C(O)C(O)C8O)C7OC7OC(C)C(O)C(O)C7O)CCC6(C)C5CCC43C)N2C1] |
| α-Tomatine | 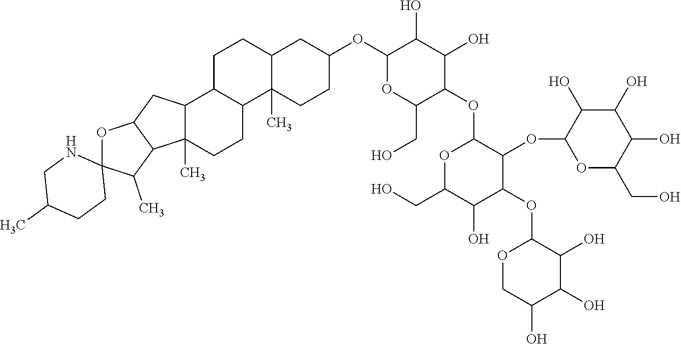 [SMILES: CC1CCC2(NC1)OC1CC3C4CCC5CC(OC6OC(CO)C(OC7OC(CO)C(O)C(OC8OCC(O)C(O)C8O)C7OC7OC(CO)C(O)C(O)C7O)C(O)C6O)CCC5(C)C4CCC3(C)C1C2C] |

*Peptide with amino acid sequence

Chelator Chel for Labeling with a Radioisotope

The chelator Chel is intended for the labeling of the active ingredient conjugate of the invention with a radioisotope selected from the group comprising ⁴⁴Sc, ⁴⁷Sc, ⁵⁵Co, ⁶²Cu, ⁶⁴Cu, ⁶⁷Cu, ⁶⁶Ga, ⁶⁷Ga, ⁶⁸Ga, ⁸⁹Zr, ⁸⁶Y, ⁹⁰Y, ⁸⁹Zr, ⁹⁰Nb, ^{99m}Tc, ¹¹¹In, ¹³⁵Sm, ¹⁵⁹Gd, ¹⁴⁹Tb, ¹⁶⁰Tb, ¹⁶¹Tb, ¹⁶⁵Er, ¹⁶⁶Dy, ¹⁶⁶Ho, ¹⁷⁵Yb, ¹⁷⁷Lu, ¹⁸⁶Re, ¹⁸⁸Re, ²¹¹At, ²¹²Pb, ²¹³Bi, ²²⁵Ac and ²³²Th. The state of the art discloses a multitude of chelators for the complexation of the above radioisotopes. Scheme 6 shows examples of chelators used in accordance with the invention.

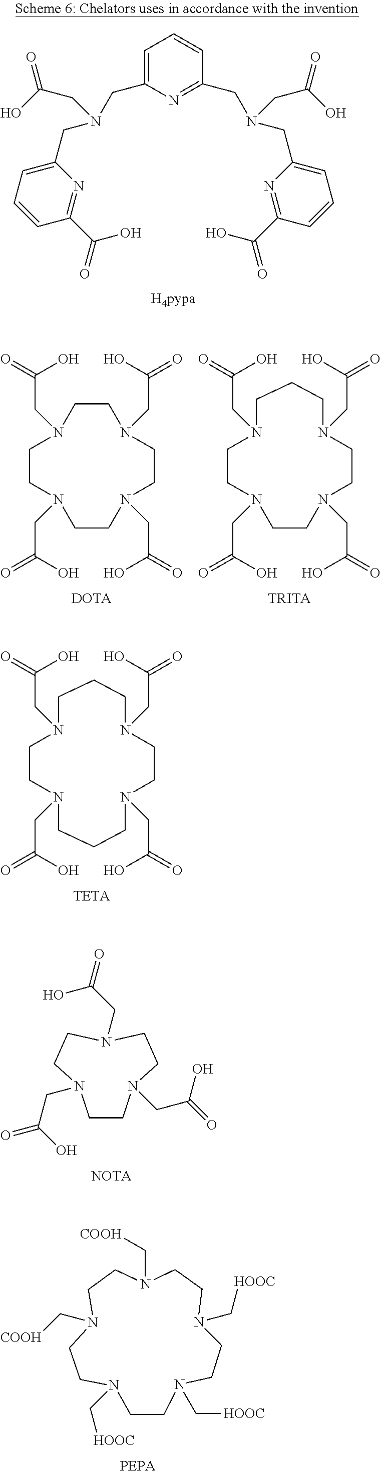

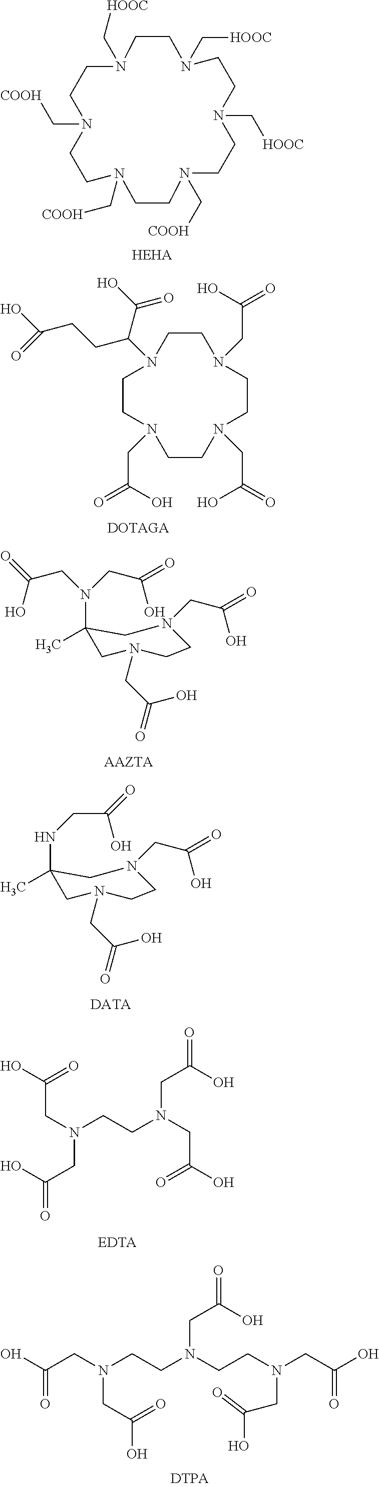

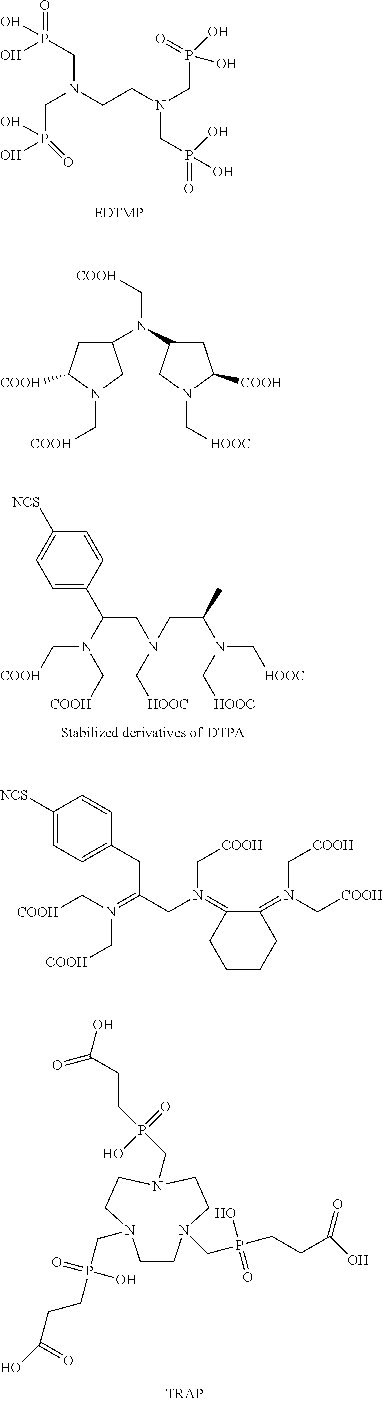

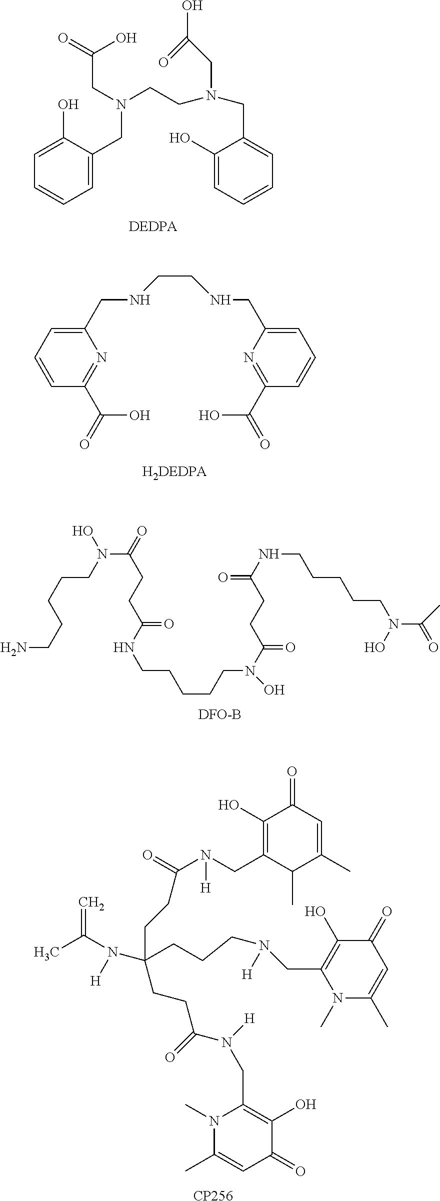

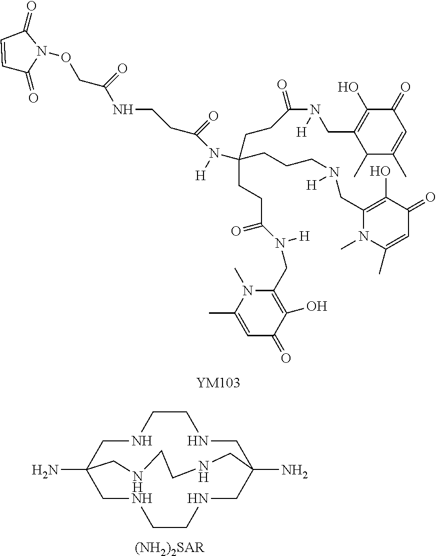

For nuclear-medical diagnosis (modality (A), (C)) and simultaneous nuclear-medical/cytotoxic theranostics (modality (B2), (D2)), the radioisotopes ⁶⁸Ga and ¹⁷⁷Lu in particular are used. The chelator DOTA, which is of good suitability for the complexation of ⁶⁸Ga and also ¹⁷⁷Lu, is preferred in accordance with the invention. For the complexation of ¹⁷⁷Lu, preference is given to using the chelator H₂pypa. The synthesis of H₄pypa is shown in scheme 7.

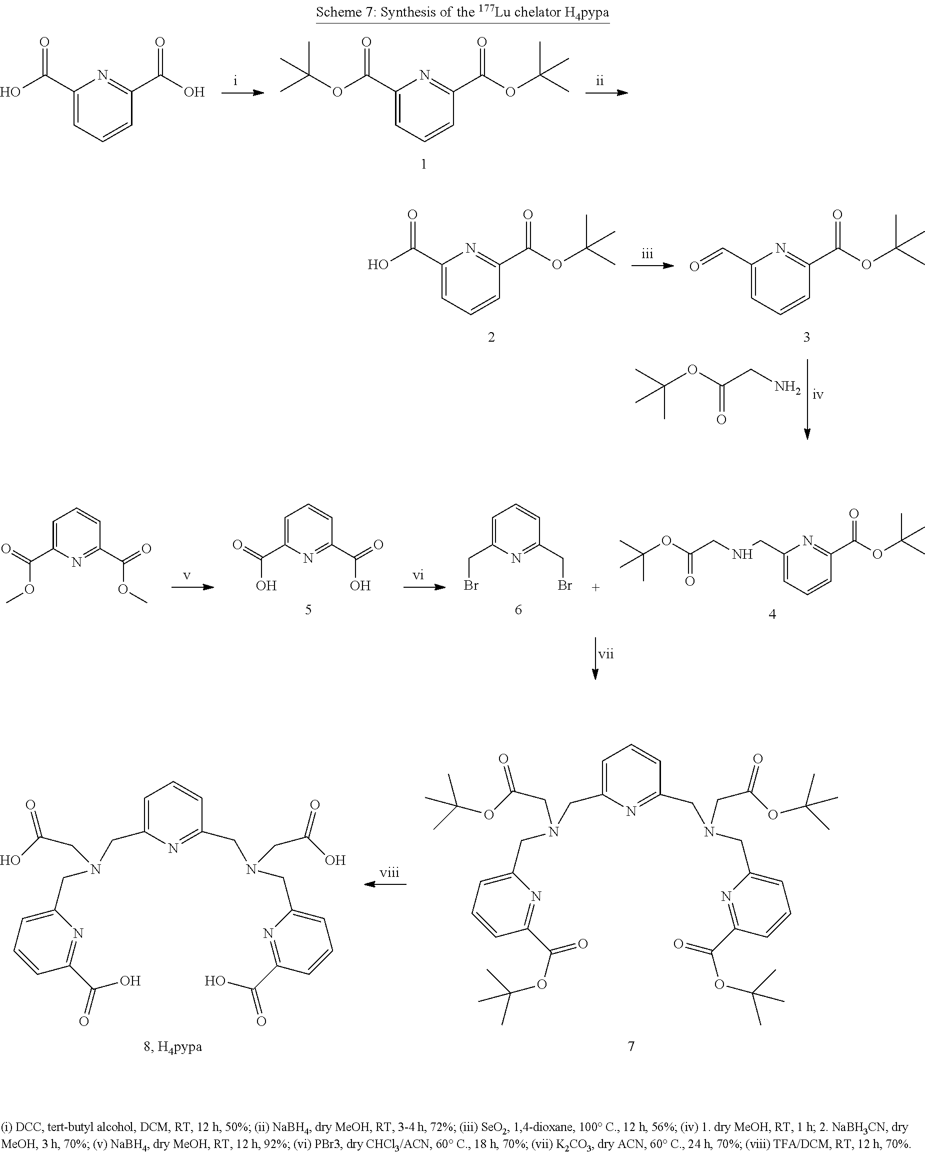

Amide Coupling

In the invention, functional groups, such as the chelator Chel, the cytotoxic compound CT, the targeting vector TV, the linkers L, L1, and the spacers S, S1, S2, are preferably conjugated by means of an amide coupling reaction. In medicinal chemistry, amide coupling, which forms the backbone of proteins, is the most commonly used reaction. A generic example of an amide coupling is shown in scheme 8.

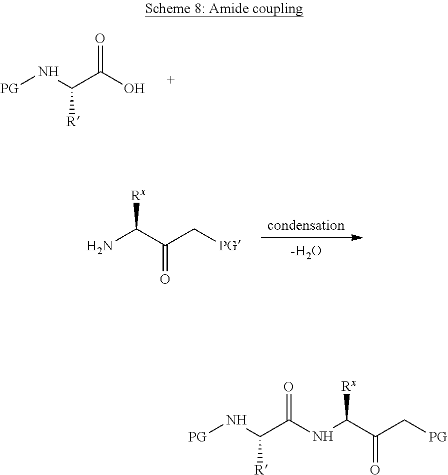

Owing to a virtually unlimited set of readily available carboxylic acid and amine derivatives, amide coupling strategies open up a simple route for the synthesis of novel compounds. The person skilled in the art is aware of numerous reagents and protocols for amide couplings. The most commonly used amide coupling strategy is based on the condensation of a carboxylic acid with an amine. For this purpose, the carboxylic acid is generally activated. Prior to the activation, remaining functional groups are protected. The reaction is effected in two steps, either in one reaction medium (single pot) with direct conversion of the activated carboxylic acid, or in two steps with isolation of activated “trapped” carboxylic acid and reaction with an amine.

The carboxylic reacts here with a coupling agent to form a reactive intermediate which can be reacted in isolated form or directly with an amine. Numerous reagents are available for carboxylic acid activation, such as acid halide (chloride, fluoride), azides, anhydrides or carbodiimides. In addition, reactive intermediates formed may be esters such as pentafluorophenyl or hydroxysuccinimido esters. Intermediates formed from acyl chlorides or azides are highly reactive. However, harsh reaction conditions and high reactivity are frequently a barrier to use for sensitive substrates or amino acids. By contrast, amide coupling strategies that utilize carbodiimides such as DCC (dicyclohexylcarbodiimide) or DIC (diisopropylcarbodiimide) open up a broad spectrum of application. Frequently, especially in the case of solid-phase synthesis, additives are used to improve reaction efficiency. Aminium salts are highly efficient peptide coupling reagents having short reaction times and minimal racemization. With some additives, for example HOBt, it is impossible to completely prevent racemization. Aminium reagents are used in an equimolar amount with the carboxylic acid in order to prevent excess reaction with the free amine of the peptide. Phosphonium salts react with carboxylate, which generally requires two equivalents of a base, for example DIEA. A significant advantage of phosphonium salts over iminium reagents is that phosphonium does not react with the free amino group of the amine component. This enables couplings in a molar ratio of acid and amine and helps to prevent the intramolecular cyclization of linear peptides and excessive use of costly amine components.

An extensive summary of reaction strategies and reagents for amide couplings can be found in the following review articles:
Analysis of Past and Present Synthetic Methodologies on Medicinal Chemistry: Where Have All the New Reactions Gone?; D. G. Brown, J. Boström; J. Med. Chem. 2016, 59, 4443-4458;
Peptide Coupling Reagents, More than a Letter Soup; A. El-Faham, F. Albericio; Chem. Rev. 2011, 111, 6557-6602;
Rethinking amide bond synthesis; V. R. Pattabiraman, J. W. Bode; Nature, Vol. 480 (2011) 22/29;
Amide bond formation: beyond the myth of coupling reagents; E. Valeur, M. Bradley; Chem. Soc. Rev., 2009, 38, 606-631.

Numerous chelators among those used in accordance with the invention, such as DOTA in particular, have one or more carboxy or amide groups. Accordingly, these chelators can be conjugated in a simple manner with the linkers L, L1 and/or spacers S, S1, S2 with the aid of one of the amide coupling strategies known in the prior art.

The cleavable group Clv present in the linkers L, L1 assures the tumor-specific release of the cytotoxic active ingredient CT and is stable in the systemic cycle, i.e. in the blood plasma. After uptake (endocytosis) into a cancer cell, the cleavable group Clv is cleaved and the cytotoxic active ingredient CT is released.

Some examples of cleavable groups Clv are given hereinafter.

Scheme 9 shows a cleavable group or a linker of the p-aminobenzoic acid-valine-citrulline type, which is cleaved by intracellular proteases, especially from the cathepsin family. Cathepsin proteases are overexpressed in prostate tumor cells.

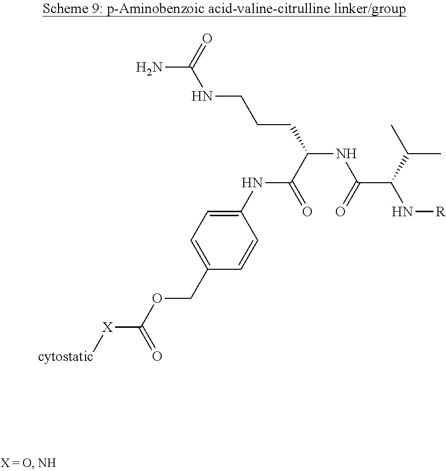

Scheme 10 shows a cleavable group or linker of the p-aminobenzoic acid-glutamate-valine-citrulline type, which is likewise cleaved by cathepsins and is notable for elevated stability in mouse serum, which constitutes a considerable advantage for preclinical studies.

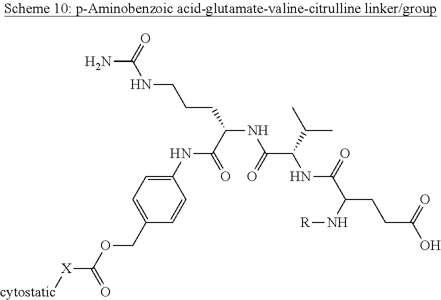

Scheme 11 shows a cleavable hydrazine group/linker which is hydrolyzed in acidic medium (pH<6.2)
as is present in tumor tissue.

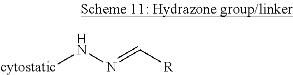

The disulfide groups/linkers shown in scheme 12 are cleaved by lysosomal glutathione (GSH: γ-L-glutamyl-L-cysteinylglycine) in a disulfide exchange reaction.

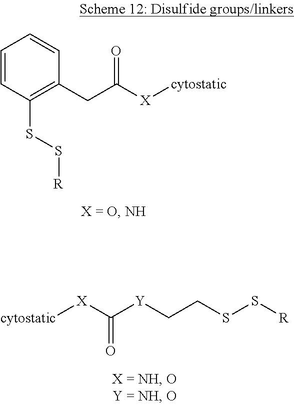

Terms used in the context of the present invention have the meaning as elucidated hereinafter.

Theranostics: Diagnosis and treatment of cancer using nuclear-medical pharmaceuticals.

Tracer: Synthetically prepared, radiolabeled substance which is used in a very small amount and is converted in the organism without affecting metabolism.

Labeling precursor: Chemical compound which contains a chelator or a functional group for labeling with a radioisotope.

Pharmaceutical kit: Single-item or multi-item pharmaceutical administration form that optionally contains one or more vessels containing one or more active ingredients that are optionally present, dissolved, suspended or emulsified in one or more carrier substances.

Vessel: Vial, septum vial, injection vial or ampoule made of glass, metal or plastic for clinical applications.

Carrier substance: Liquid or solid substance that serves as pharmaceutical carrier for an active pharmaceutical ingredient and generally does not have any pharmaceutical activity.

Smart drug delivery system (SDDS): Chemical compound comprising a cytotoxic active ingredient, a cleavable linker for release of the cytotoxic active ingredient, and a targeting vector for accumulation in tumor tissue, and optionally a further linker or spacer and a chelator for labeling with a radioisotope.

Residue of a chelator: Chelator as part of a chemical compound, especially as part of an SDDS compound.

Target: Biological target structure, especially (membrane-bound) receptor, protein or antibody in a living organism to which a targeting vector binds.

Targeting vector: Chemical group or residue that serves as ligand, agonist, antagonist or inhibitor for a target and has high binding affinity for that target.

Radiopharmaceutical: Radiolabeled chemical compound or labeling precursor complexed with a radioisotope for nuclear-medical diagnosis and theranostics.

Linker: Structural unit, group or radical which comprises a biologically cleavable subgroup or sub unit and via which a targeting vector, a cytotoxic active ingredient or a chelator is bound to a further structural unit.

Cleavable group: Structural unit, group or residue which is cleaved by enzymes or molecules present in the cytoplasm, in endosomes or lysosomes.

Spacer: Structural unit which functions as spacer between a targeting vector and a chelator and counteracts steric hindrance of the targeting vector by the chelator. In particular appropriate embodiments of the invention, the spacer comprises a cleavable group and is designed as a linker.

Active ingredient conjugate: Compound comprising a cytotoxic active ingredient, a targeting vector and a cleavable linker.

Dual active ingredient conjugate: Compound comprising a cytotoxic active ingredient, a targeting vector, a chelator, a linker and a spacer.

### EXAMPLES

### Example 1: Dual Active Ingredient Conjugates

Schemes 13 to 22 show examples of inventive dual active ingredient conjugates according to FIG. 1a, comprising a targeting vector, a chelator for labeling with a radioisotope and a cytotoxic active ingredient.

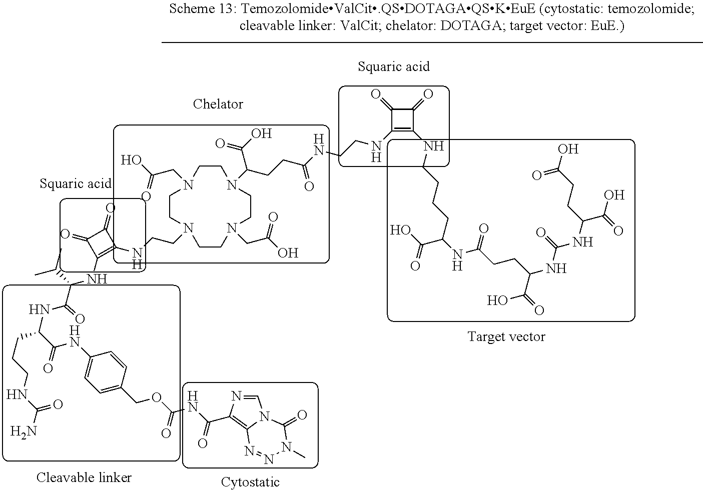 [SMILES: CC(C)[C@H](Nc1c(NCCN2CCN(CC(=O)O)CCN(C(CCC(=O)NCCNc3c(NCCCCC(NC(=O)CCC(NC(=O)NC(CCC(=O)O)C(=O)O)C(=O)O)C(=O)O)c(=O)c3=O)C(=O)O)CCN(CC(=O)O)CC2)c(=O)c1=O)C(=O)N[C@@H](CCCNC(N)=O)C(=O)Nc1ccc(COC(=O)NC(=O)c2ncn3c(=O)n(C)nnc23)cc1]

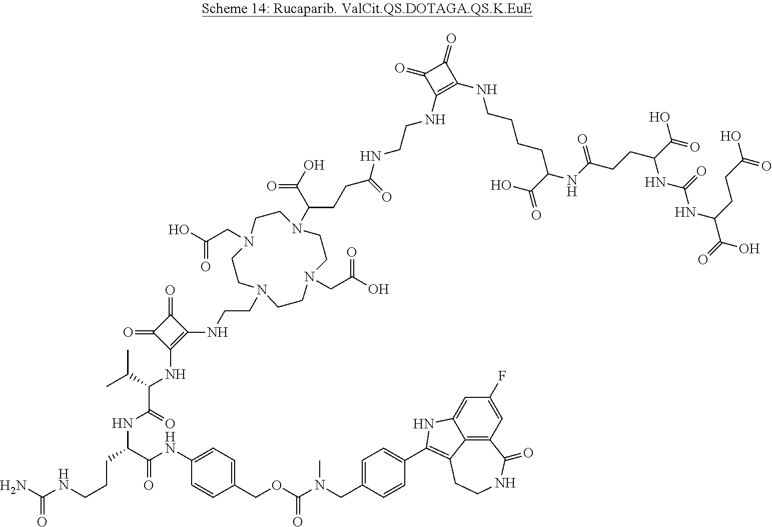 [SMILES: CC(C)[C@H](Nc1c(NCCN2CCN(CC(=O)O)CCN(C(CCC(=O)NCCNc3c(NCCCCC(NC(=O)CCC(NC(=O)NC(CCC(=O)O)C(=O)O)C(=O)O)C(=O)O)c(=O)c3=O)C(=O)O)CCN(CC(=O)O)CC2)c(=O)c1=O)C(=O)N[C@@H](CCCNC(N)=O)C(=O)Nc1ccc(COC(=O)N(C)Cc2ccc(-c3[nH]c4cc(F)cc5c4c3CCNC5=O)cc2)cc1]

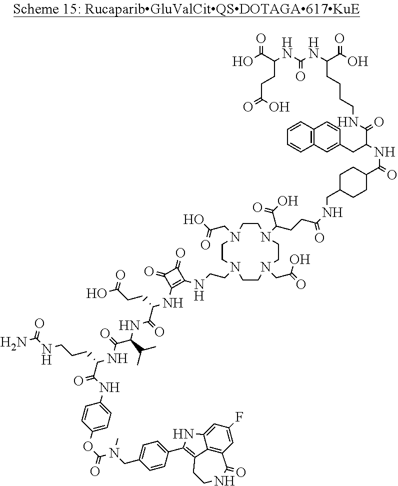 [SMILES: CC(C)[C@H](NC(=O)[C@H](CCC(=O)O)Nc1c(NCCN2CCN(CC(=O)O)CCN(C(CCC(=O)NCC3CCC(C(=O)NC(Cc4ccc5ccccc5c4)C(=O)NCCCCC(NC(=O)NC(CCC(=O)O)C(=O)O)C(=O)O)CC3)C(=O)O)CCN(CC(=O)O)CC2)c(=O)c1=O)C(=O)N[C@@H](CCCNC(N)=O)C(=O)Nc1ccc(OC(=O)N(C)Cc2ccc(-c3[nH]c4cc(F)cc5c4c3CCNC5=O)cc2)cc1]

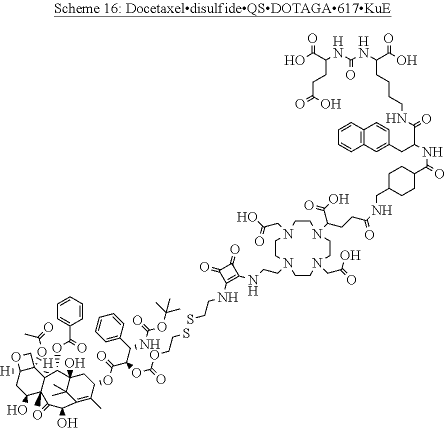 [SMILES: CC(=O)O[C@@]12CO[C@@H]1C[C@H](O)[C@@]1(C)C(=O)[C@H](O)C3=C(C)[C@@H](OC(=O)[C@H](OC(=O)OCCSSCCNc4c(NCCN5CCN(CC(=O)O)CCN(C(CCC(=O)NCC6CCC(C(=O)NC(Cc7ccc8ccccc8c7)C(=O)NCCCCC(NC(=O)NC(CCC(=O)O)C(=O)O)C(=O)O)CC6)C(=O)O)CCN(CC(=O)O)CC5)c(=O)c4=O)[C@@H](NC(=O)OC(C)(C)C)c4ccccc4)C[C@@](O)([C@@H](OC(=O)c4ccccc4)[C@H]21)C3(C)C]

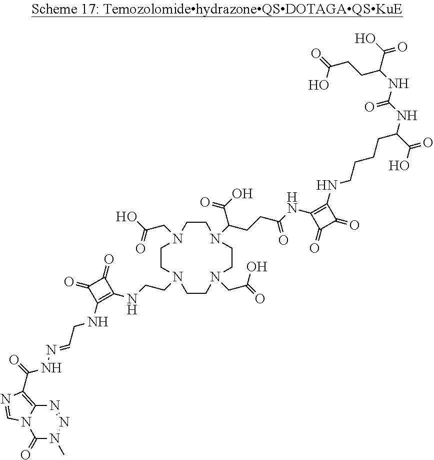 [SMILES: Cn1nnc2c(C(=O)N/N=C/CNc3c(NCCN4CCN(CC(=O)O)CCN(C(CCC(=O)Nc5c(NCCCCC(NC(=O)NC(CCC(=O)O)C(=O)O)C(=O)O)c(=O)c5=O)C(=O)O)CCN(CC(=O)O)CC4)c(=O)c3=O)ncn2c1=O]

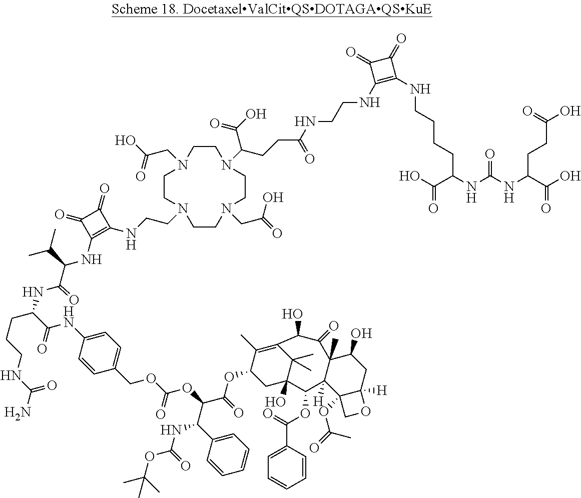 [SMILES: CC(=O)O[C@@]12CO[C@@H]1C[C@H](O)[C@@]1(C)C(=O)[C@H](O)C3=C(C)[C@@H](OC(=O)[C@H](OC(=O)OCc4ccc(NC(=O)[C@H](CCCNC(N)=O)NC(=O)[C@H](Nc5c(NCCN6CCN(CC(=O)O)CCN(C(CCC(=O)NCCNc7c(NCCCCC(NC(=O)NC(CCC(=O)O)C(=O)O)C(=O)O)c(=O)c7=O)C(=O)O)CCN(CC(=O)O)CC6)c(=O)c5=O)C(C)C)cc4)[C@@H](NC(=O)OC(C)(C)C)c4ccccc4)C[C@@](O)([C@@H](OC(=O)c4ccccc4)[C@H]21)C3(C)C]

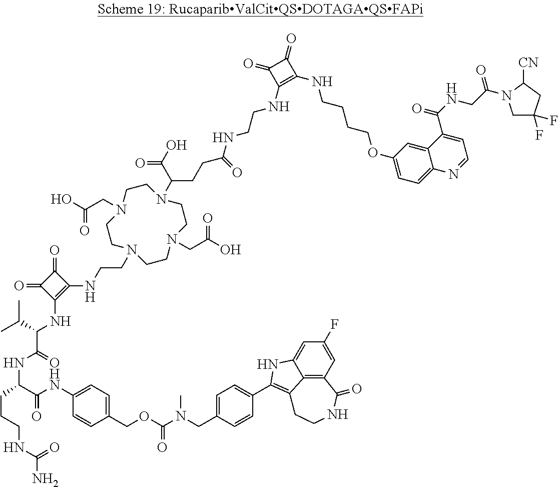 [SMILES: CC(C)[C@H](Nc1c(NCCN2CCN(CC(=O)O)CCN(C(CCC(=O)NCCNc3c(NCCCCOc4ccc5nccc(C(=O)NCC(=O)N6CC(F)(F)CC6C#N)c5c4)c(=O)c3=O)C(=O)O)CCN(CC(=O)O)CC2)c(=O)c1=O)C(=O)N[C@@H](CCCNC(N)=O)C(=O)Nc1ccc(COC(=O)N(C)Cc2ccc(-c3[nH]c4cc(F)cc5c4c3CCNC5=O)cc2)cc1]

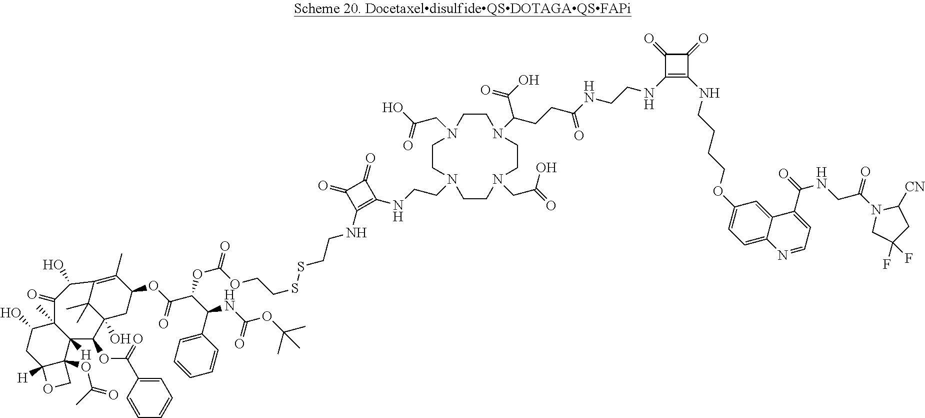 [SMILES: CC(=O)O[C@@]12CO[C@@H]1C[C@H](O)[C@@]1(C)C(=O)[C@H](O)C3=C(C)[C@@H](OC(=O)[C@H](OC(=O)OCCSSCCNc4c(NCCN5CCN(CC(=O)O)CCN(C(CCC(=O)NCCNc6c(NCCCCOc7ccc8nccc(C(=O)NCC(=O)N9CC(F)(F)CC9C#N)c8c7)c(=O)c6=O)C(=O)O)CCN(CC(=O)O)CC5)c(=O)c4=O)[C@@H](NC(=O)OC(C)(C)C)c4ccccc4)C[C@@](O)([C@@H](OC(=O)c4ccccc4)[C@H]21)C3(C)C]

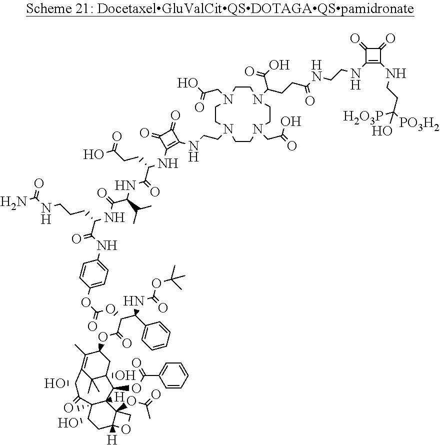

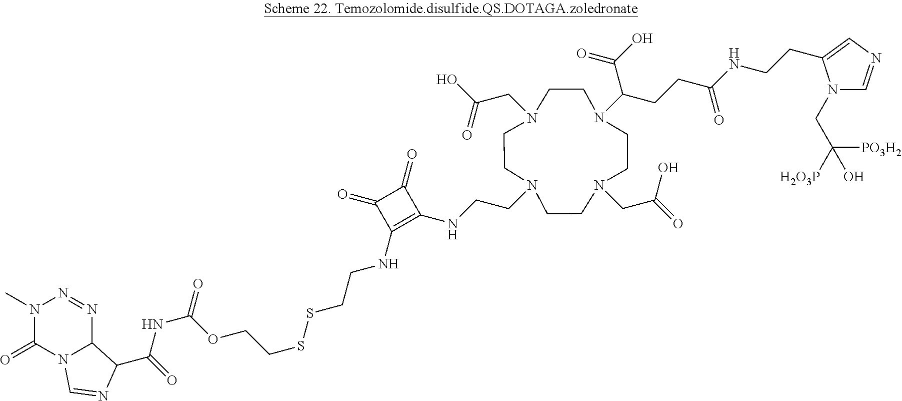

### Example 2: Dual Active Ingredient Conjugates According to FIG. 1b

Schemes 23, 24, 25 and 26 show examples of inventive dual active ingredient conjugates according to FIG. 1b, comprising a targeting vector, a chelator for labeling with a radioisotope, a cleavable linker and a cytotoxic active ingredient.

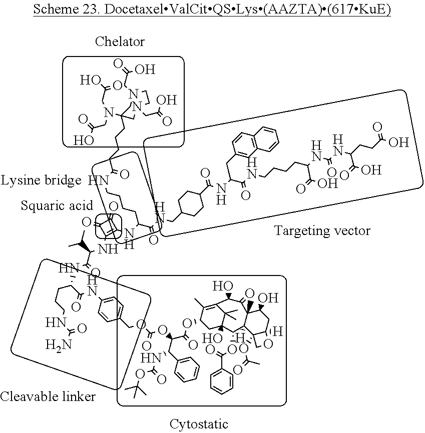 [SMILES: CC(=O)O[C@@]12CO[C@@H]1C[C@H](O)[C@@]1(C)C(=O)[C@H](O)C3=C(C)[C@@H](OC(=O)[C@H](OC(=O)OCc4ccc(NC(=O)[C@H](CCCNC(N)=O)NC(=O)[C@H](Nc5c(NC(CCCCNC(=O)CCCCC6(N(CC(=O)O)CC(=O)O)CN(CC(=O)O)CCN(CC(=O)O)C6)C(=O)NCC6CCC(C(=O)NC(Cc7ccc8ccccc8c7)C(=O)NCCCCC(NC(=O)NC(CCC(=O)O)C(=O)O)C(=O)O)CC6)c(=O)c5=O)C(C)C)cc4)[C@@H](NC(=O)OC(C)(C)C)c4ccccc4)C[C@@](O)([C@@H](OC(=O)c4ccccc4)[C@H]21)C3(C)C]

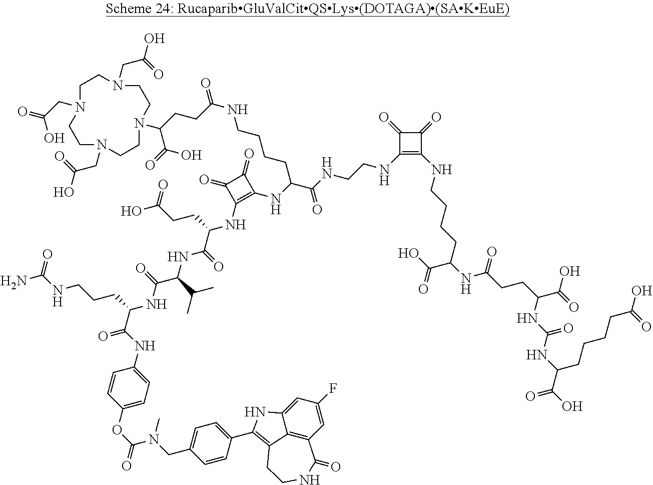 [SMILES: CC(C)[C@H](NC(=O)[C@H](CCC(=O)O)Nc1c(NC(CCCCNC(=O)CCC(C(=O)O)N2CCN(CC(=O)O)CCN(CC(=O)O)CCN(CC(=O)O)CC2)C(=O)NCCNc2c(NCCCCC(NC(=O)CCC(NC(=O)NC(CCCCC(=O)O)C(=O)O)C(=O)O)C(=O)O)c(=O)c2=O)c(=O)c1=O)C(=O)N[C@@H](CCCNC(N)=O)C(=O)Nc1ccc(OC(=O)N(C)Cc2ccc(-c3[nH]c4cc(F)cc5c4c3CCNC5=O)cc2)cc1]

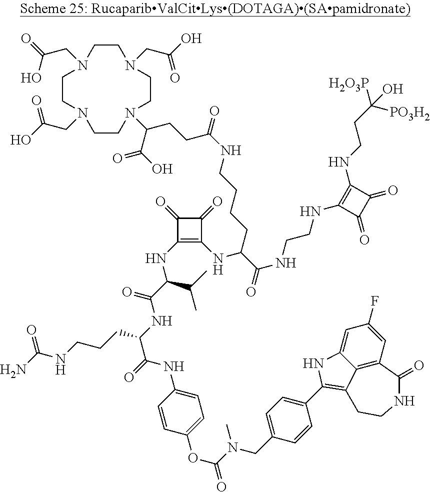

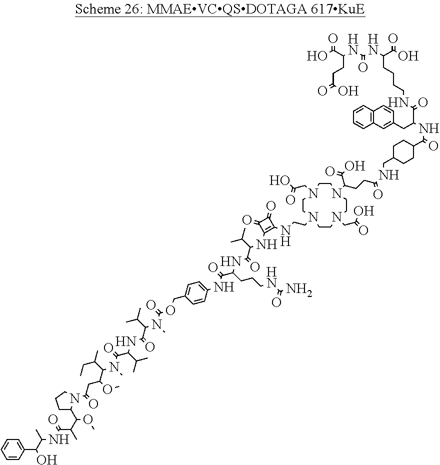 [SMILES: CCC(C)C(C(CC(=O)N1CCCC1C(OC)C(C)C(=O)NC(C)C(O)c1ccccc1)OC)N(C)C(=O)C(NC(=O)C(C(C)C)N(C)C(=O)OCc1ccc(NC(=O)C(CCCNC(N)=O)NC(=O)C(Nc2c(NCCN3CCN(CC(=O)O)CCN(C(CCC(=O)NCC4CCC(C(=O)NC(Cc5ccc6ccccc6c5)C(=O)NCCCCC(NC(=O)NC(CCC(=O)O)C(=O)O)C(=O)O)CC4)C(=O)O)CCN(CC(=O)O)CC3)c(=O)c2=O)C(C)C)cc1)C(C)C]

### Example 3: Active Ingredient Conjugates According to FIG. 1d

Schemes 27, 28, 29 and 30 show examples of inventive active ingredient conjugates according to FIG. 1d, comprising a targeting vector, a cleavable linker and a cytotoxic active ingredient.

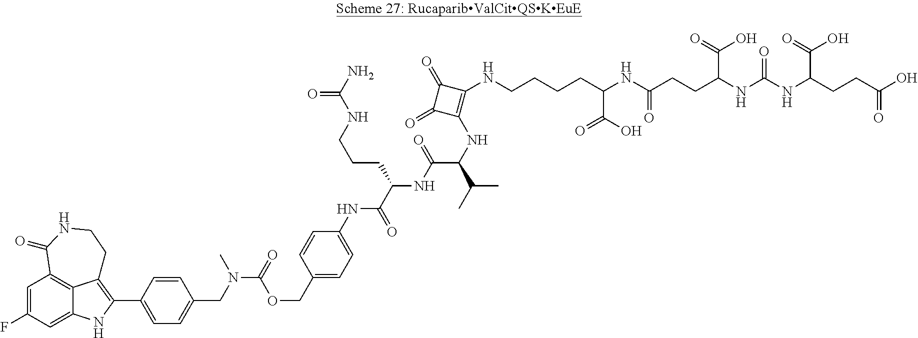 [SMILES: CC(C)[C@H](Nc1c(NCCCCC(NC(=O)CCC(NC(=O)NC(CCC(=O)O)C(=O)O)C(=O)O)C(=O)O)c(=O)c1=O)C(=O)N[C@@H](CCCNC(N)=O)C(=O)Nc1ccc(COC(=O)N(C)Cc2ccc(-c3[nH]c4cc(F)cc5c4c3CCNC5=O)cc2)cc1]

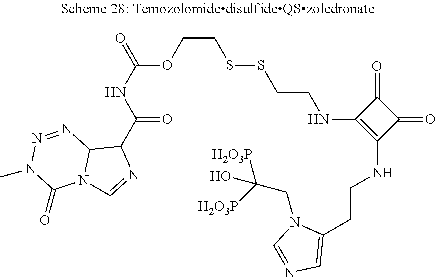

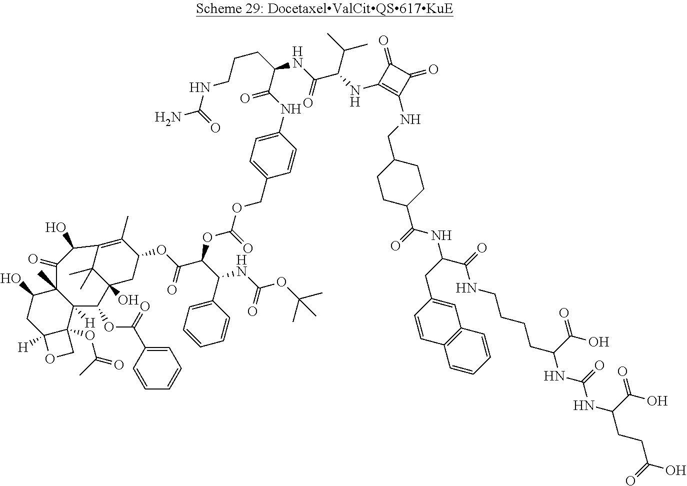 [SMILES: CC(=O)O[C@]12CO[C@H]1C[C@@H](O)[C@]1(C)C(=O)[C@@H](O)C3=C(C)[C@H](OC(=O)[C@@H](OC(=O)OCc4ccc(NC(=O)[C@@H](CCCNC(N)=O)NC(=O)[C@@H](Nc5c(NCC6CCC(C(=O)NC(Cc7ccc8ccccc8c7)C(=O)NCCCCC(NC(=O)NC(CCC(=O)O)C(=O)O)C(=O)O)CC6)c(=O)c5=O)C(C)C)cc4)[C@H](NC(=O)OC(C)(C)C)c4ccccc4)C[C@](O)([C@H](OC(=O)c4ccccc4)[C@@H]21)C3(C)C]

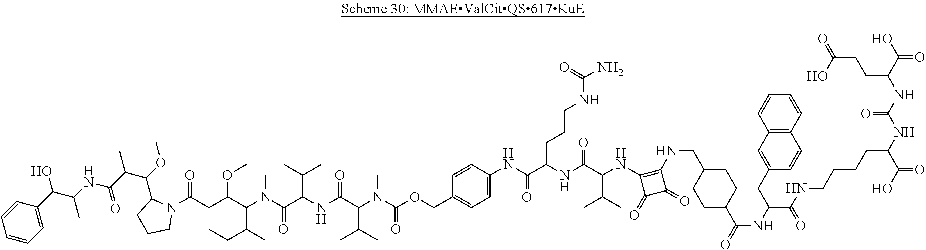 [SMILES: CCC(C)C(C(CC(=O)N1CCCC1C(OC)C(C)C(=O)NC(C)C(O)c1ccccc1)OC)N(C)C(=O)C(NC(=O)C(C(C)C)N(C)C(=O)OCc1ccc(NC(=O)C(CCCNC(N)=O)NC(=O)C(Nc2c(NCC3CCC(C(=O)NC(Cc4ccc5ccccc5c4)C(=O)NCCCCC(NC(=O)NC(CCC(=O)O)C(=O)O)C(=O)O)CC3)c(=O)c2=O)C(C)C)cc1)C(C)C]

### Example 4: Synthesis Strategy for PSMA Labeling Precursors

In the synthesis of the active ingredient conjugates of the invention, preference is given to using squaric diesters. In this way, it is possible to prepare a multitude of in some cases very complex active ingredient conjugates by means of simple reactions. Squaric diesters are notable for their selective reaction with amines, such that protecting groups are not required for the coupling of chelators, linkers, spacers and targeting vectors. Moreover, the coupling reaction is controllable via the pH.

First, a targeting vector for PSMA is synthesized (see scheme 31a) and, after purification, in aqueous medium at pH=7, reacted with squaric diester to give a precursor for coupling with a chelator (see scheme 32). Alternatively, the coupling can also be conducted in an organic medium with triethylamine as base.

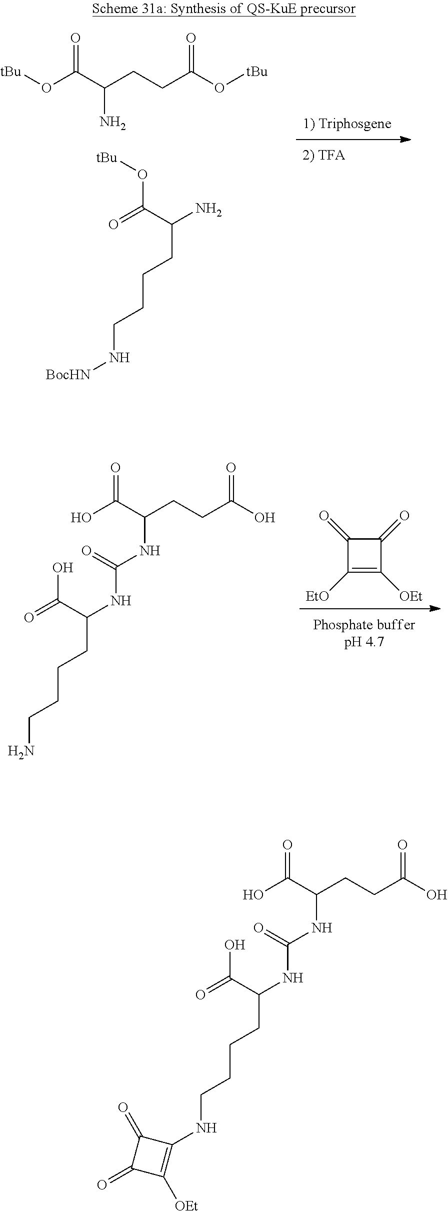

The target vector synthesized for PSMA by means of a known method is, for example, the PSMA inhibitor L-lysine-urea-L-glutamate (KuE) (cf. scheme 31b). This involves reacting a polymer resin-bound and tert-butyloxycarbonyl-protected (tert-butyl-protected) lysine with di-tert-butyl-protected glutamic acid. After the protected glutamic acid has been activated by triphosgene and coupled to the solid-phase-bound lysine, L-lysine-urea-L-glutamate (KuE) is eliminated by means of TFA and at the same time fully deprotected. The product can subsequently be separated from free lysine by means of semipreparative HPLC with a yield of 71%.

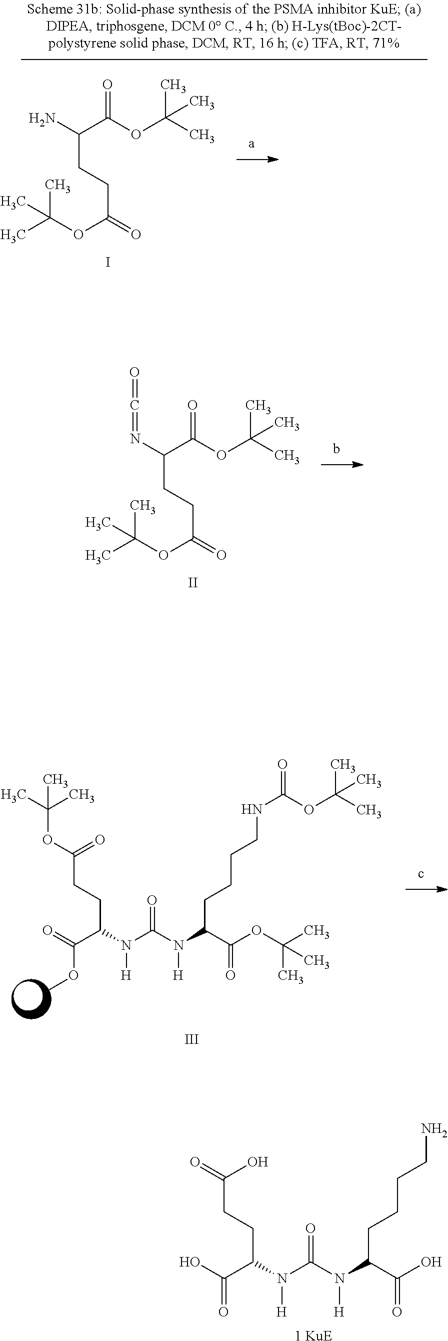

The PSMA inhibitor KuE (1) can then be coupled by means of diethyl squarate as coupling reagent to a labeling precursor (cf. scheme 32). The coupling of KuE (1) to squaric diester is effected in 0.5 M phosphate buffer at a pH of pH 7. After the two reactants have been added, the pH has to be readjusted with sodium hydroxide solution (1 M) since the buffer capacity of the phosphate buffer is insufficient. At pH 7, the single amidation of the acid proceeds at room temperature with a short reaction time. KuE-QS (2) is obtained after HPLC purification with an overall yield of 16%.

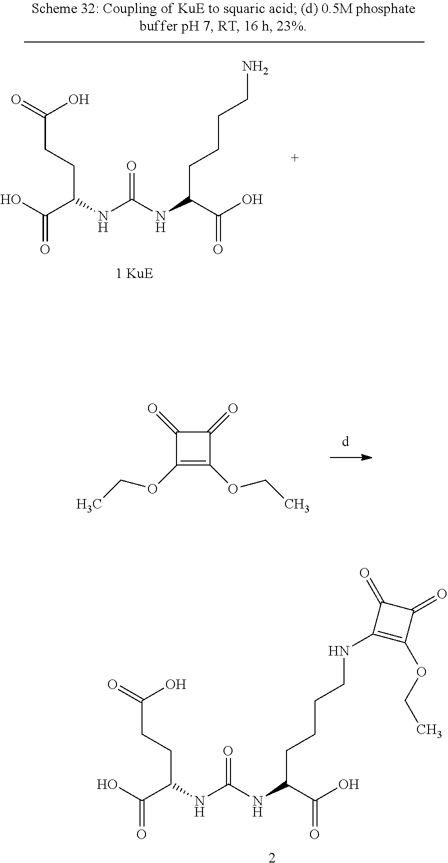

The KuE squaric acid monoester thus obtained is storable and can be used as a building block for further syntheses.

### Example 5: Solid-Phase-Based Synthesis of the KuE Unit and of the PSMA-617 Linker

The conjugation of the glutamate-urea-lysine binding motif KuE to an aromatic linker unit was effected by a solid-phase peptide synthesis described by Benesova et al. (Linker Modification Strategies To Control the Prostate-Specific Membrane Antigen (PSMA)-Targeting and Pharmacokinetic Properties of DOTA-Conjugated PSMA Inhibitors; J Med Chem, 2016, 59, 1761-1775). The synthesis reported by Benesova et al. was modified slightly (cf. scheme 33).

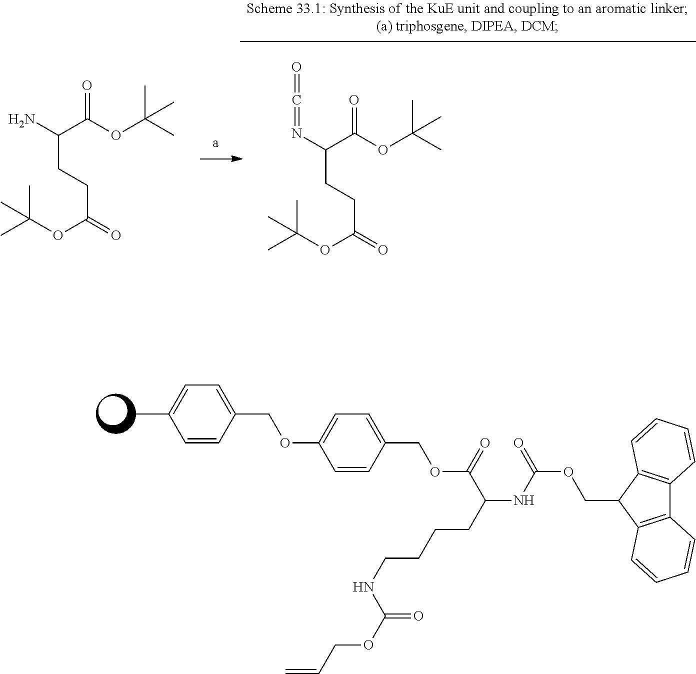 [SMILES: CC(C)(C)OC(=O)CCC(N)C(=O)OC(C)(C)C ; CC(C)(C)OC(=O)CCC(N=C=O)C(=O)OC(C)(C)C ; C=CCOC(=O)NCCCCC(NC(=O)OCC1c2ccccc2-c2ccccc21)C(=O)OCc1ccc(OCc2ccc(C)cc2)cc1]

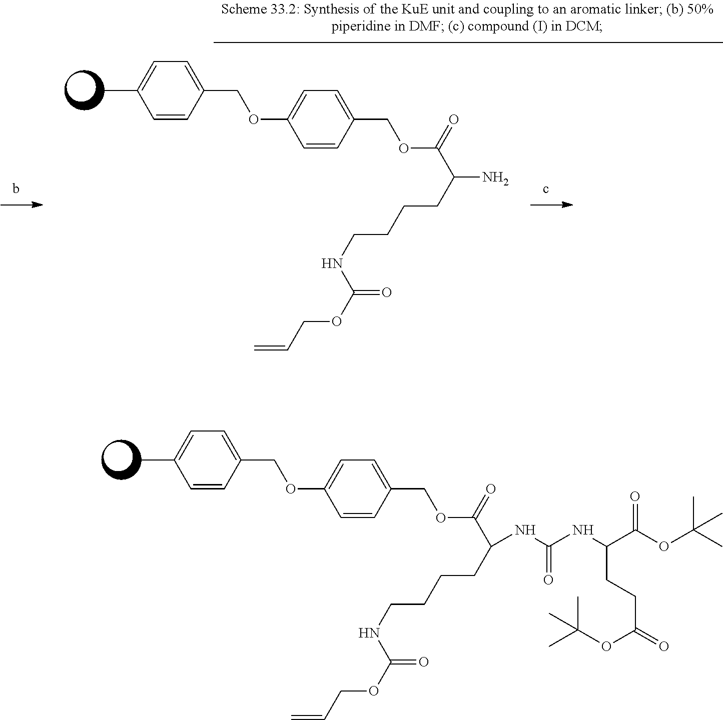 [SMILES: C=CCOC(=O)NCCCCC(N)C(=O)OCc1ccc(OCc2ccc(C)cc2)cc1 ; C=CCOC(=O)NCCCCC(NC(=O)NC(CCC(=O)OC(C)(C)C)C(=O)OC(C)(C)C)C(=O)OCc1ccc(OCc2ccc(C)cc2)cc1]

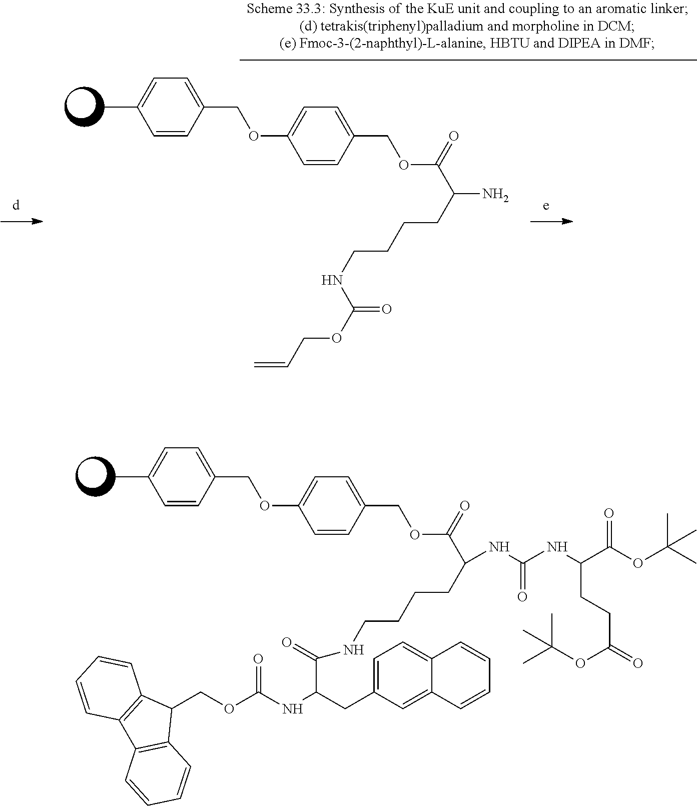 [SMILES: C=CCOC(=O)NCCCCC(N)C(=O)OCc1ccc(OCc2ccc(C)cc2)cc1 ; Cc1ccc(COc2ccc(COC(=O)C(CCCCNC(=O)C(Cc3ccc4ccccc4c3)NC(=O)OCC3c4ccccc4-c4ccccc43)NC(=O)NC(CCC(=O)OC(C)(C)C)C(=O)OC(C)(C)C)cc2)cc1]

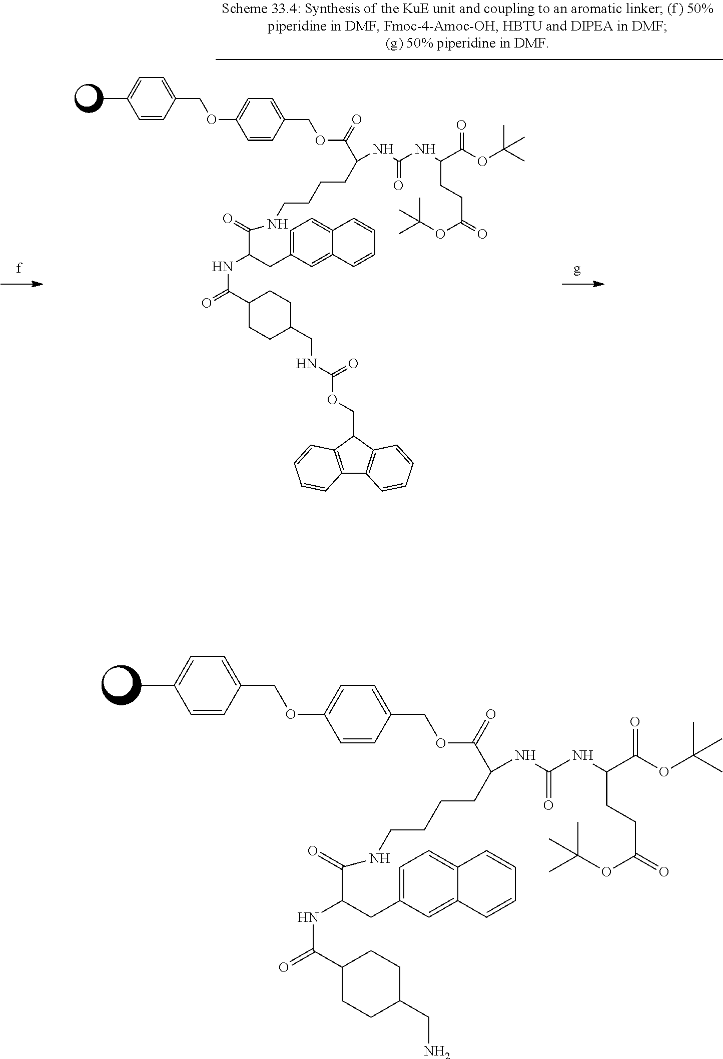

### Example 6: Synthesis of the Coupling-Capable DOTAGA Chelator and Coupling Thereof to the PSMA-617 Target Vector-Linker Unit

The synthesis proceeds from commercially available DO2A(tBu)-GABz, which is functionalized on the secondary amine with a Boc-protected amino group (cf. scheme 34).

This enables the late introduction of the cytostatic-linker unit.

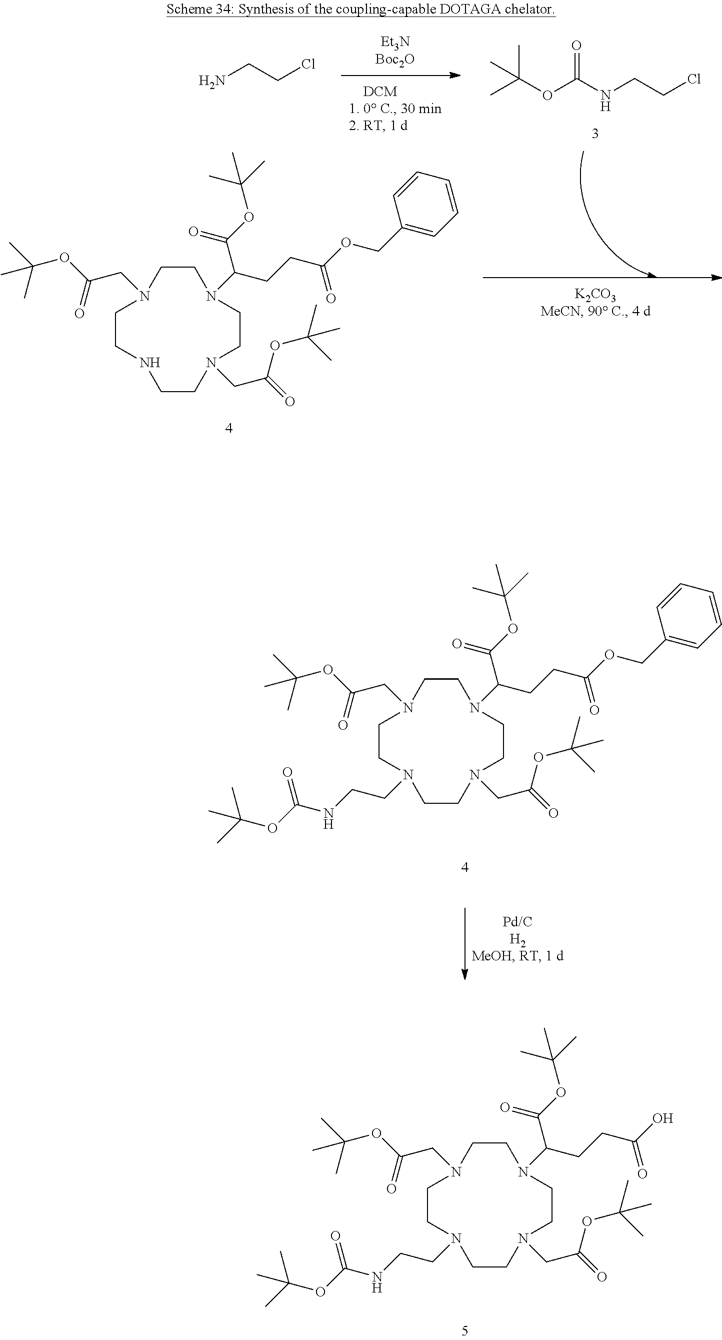

The benzyl protecting group of the glutaric acid side chain of the DOTAGA(COOtBu)3(NHBoc)-GABz 4 is reductively removed in order to enable coupling to the PSMA target vector via a linker.

Then the linker-PSMA conjugate is coupled to the chelator 6 by means of amide coupling.

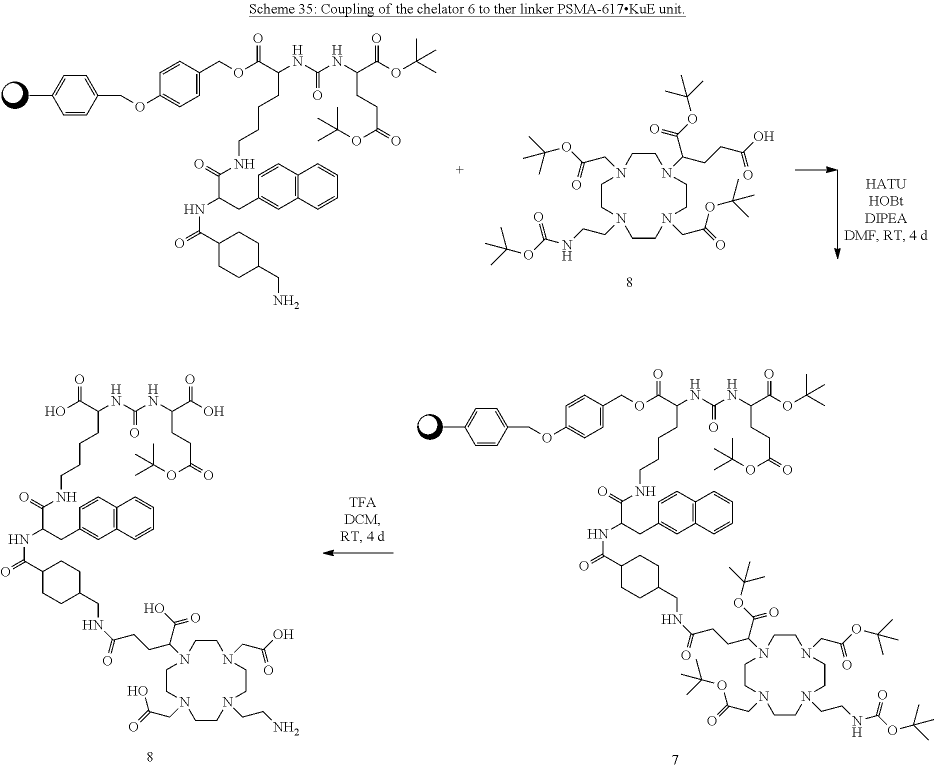

The coupling of the chelator 6 to the KuE-bound linker is described in scheme 35. The protected PSMA617 derivative 7 obtained by the amide coupling is deprotected with the aid of trifluoroacetic acid (TFA) and separated from the solid phase. The overall yield of the two-stage synthesis after HPLC purification is 6%.

### Example 7: Synthesis of the Inventive Compound MMAE.ValCit.QS.617.KuE

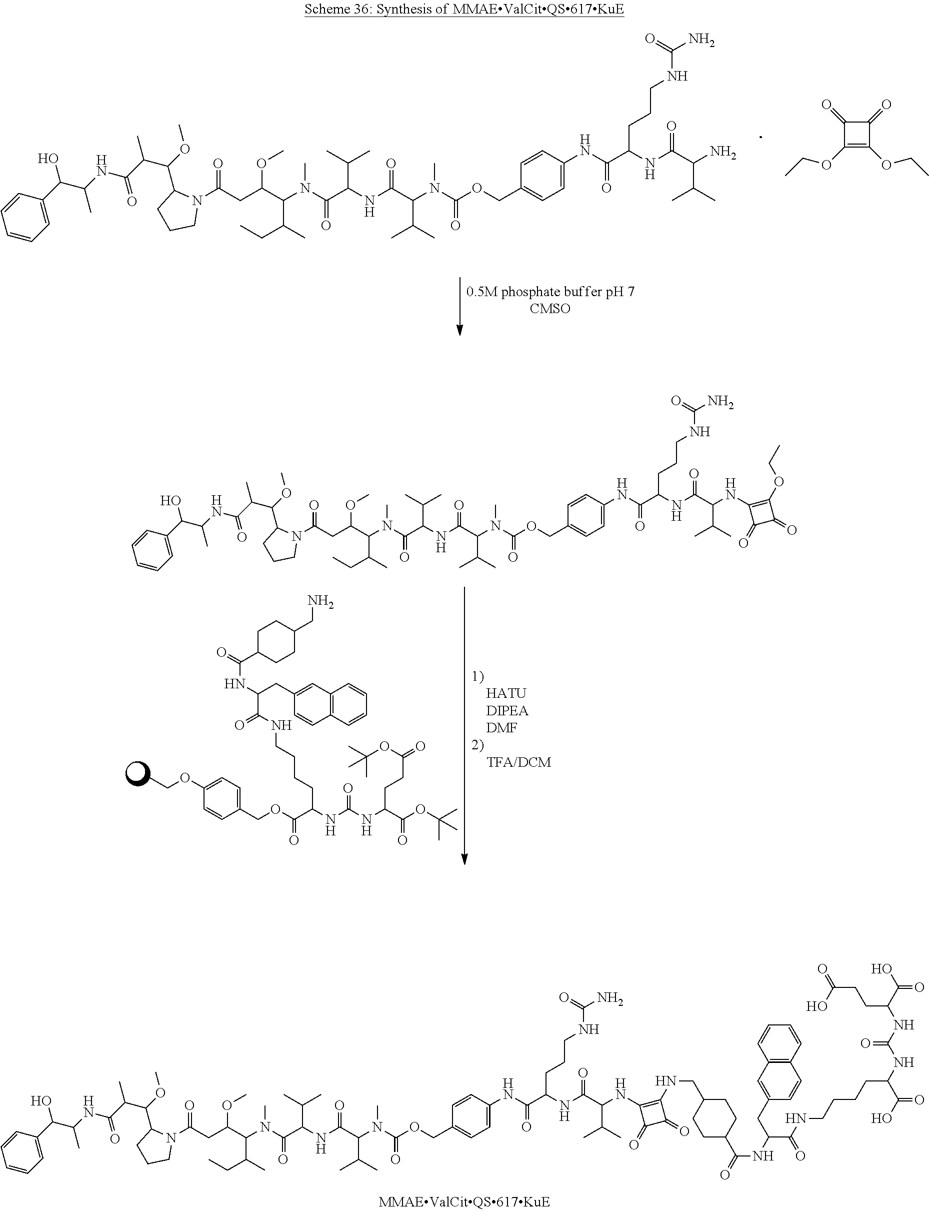

The synthesis of the compound MMAE.ValCit.QS.617.KuE proceeds from commercially available MMAE.ValCit, which is coupled to diethyl squarate at a pH of 7 in phosphate buffer (0.5 M) with addition of DMSO (cf. scheme 36). This is followed by solid-phase-based coupling of the MMAE.ValCit.QS unit and the 617.KuE-linker-target vector unit in ethanol with addition of 2% triethylamine. After HPLC purification, the yield of the synthesis was 43%.

### Example 8: Radiolabeling

For the radiolabeling of the PSMA labeling precursors, ⁶⁸Ga was eluted from an ITG Ge/Ga generator with 0.05 M HCl and process by means of aqueous ethanol elution through a cation exchange column. According to the chelator, radiolabeling is effected at pH values between 3.5 and 5.5 and temperatures between 25° C. and 95° C. The progress of the reaction was recorded by means of HPLC and IPTC in order to ascertain the kinetic parameters of the reaction.

### Example 9: Squaric Acid as Complexing Aid

For clinical use, it is very important that complexation proceeds efficiently at low temperature. Squaric acid complexes free metals and can thus protect the chelator site from non-specific coordination. This effect has been observed in the case of radiolabeling of TRAP.QS at different temperatures. TRAP complexes quantitatively at room temperature. By contrast, under the same conditions, in the case of TRAP.QS, an RCY value of only 50% was measured. If the temperature is increased, there is a rise in the labeling yield of TRAP.QS to quantitative values. This shows the influence that squaric acid has on complexation. This effect, illustrated in scheme 37, enables the stable complexation of metals having a high coordination number, for example zirconium, with the aid of the chelator AAZTA.QS.

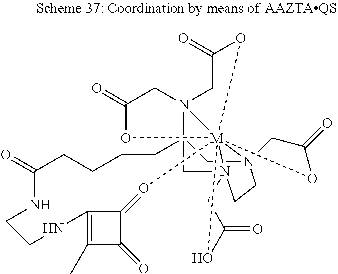

In appropriate embodiments of the pharmaceutical kit of the invention, the first, second and/or third compound contains one or more squaric acid radicals QS. The use of squaric diesters allows coupling reactions to be simplified considerably.

### Example 10a: Squaric Acid as Affinity Promoter

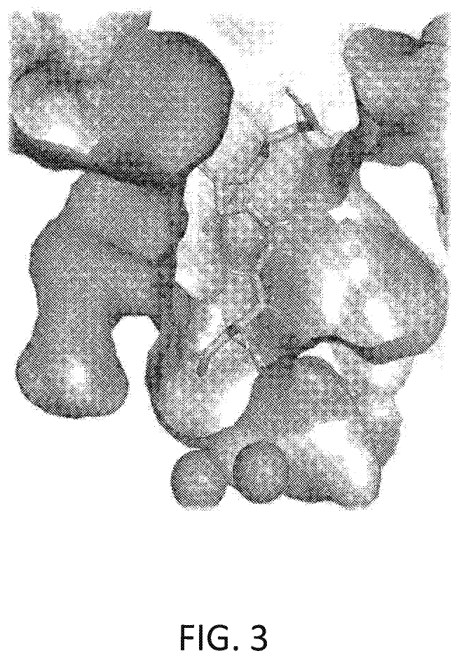

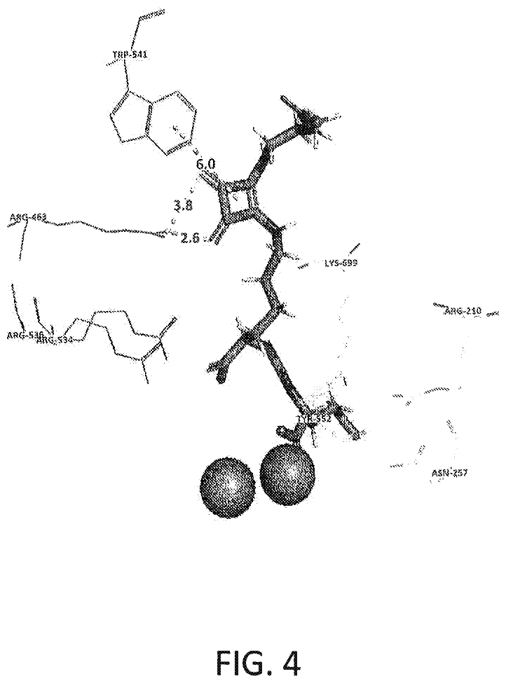

Moreover, the inventors have found that, surprisingly, the incorporation of squaric acid groups QS improves pharmacological properties and increases the binding affinity of PSMA-specific targeting vectors. The inventors suspect that the binding affinity is increased by ionic interaction of the squaric acid group QS with ARG463. To verify this hypothesis, docking studies were conducted. FIGS. 3 and 4 show the arrangements favored on the basis of the docking studies. ARG463 is located in what is called the arginine patch of PSMA. A further putative mechanism of action is based on hydrogen bonds to Trp541, which increase affinity for the arene binding pocket of PSMA.

The squaric acid group interacts with Arg463 in the arginine-rich region (dark region) and with Trp541 in the arene binding pocket. The dotted light-colored lines represent the distance in Å. The zinc ions present in the active binding pocket are shown as spheres. The structure data are based on the structure, determined by means of x-ray diffraction, of PSMA in complex with PSMA 1007 (PDB 5O5T).

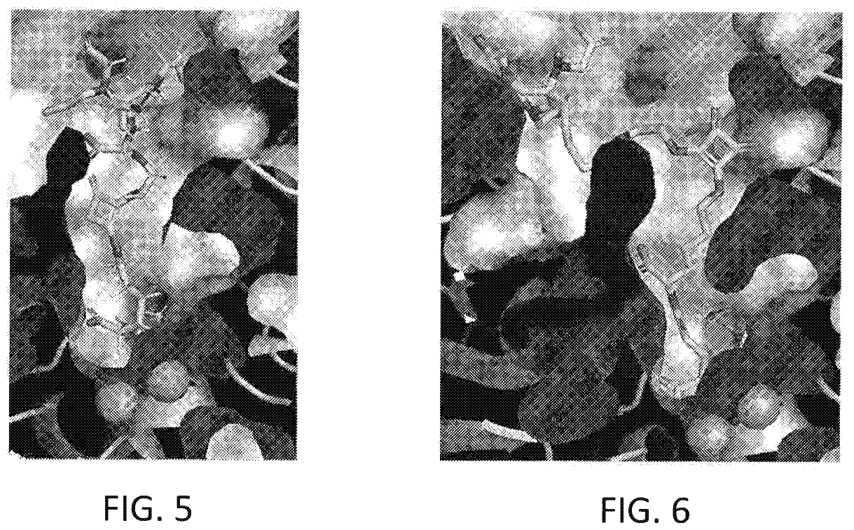

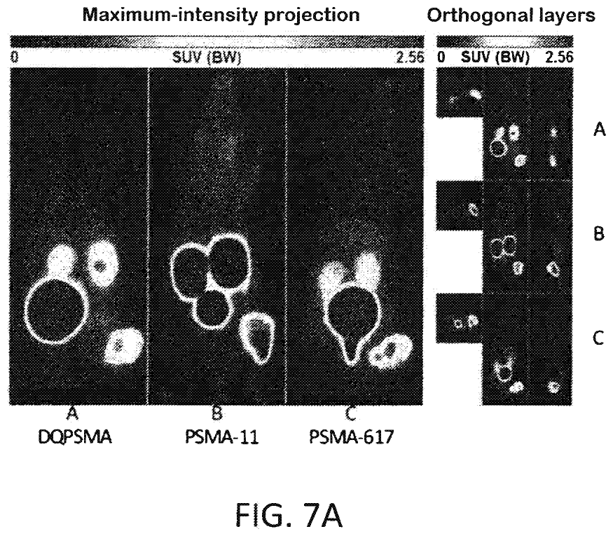

FIG. 5 shows the putative binding mode of AAZTA.QS.KuE in the binding pocket of PSMA. The AAZTA chelator project out of the PSMA pocket. The QS linker interacts with the hydrophobic portion of the binding pocket. The binding motif is in the pharmacophore portion of the pocket and is complexed by the two zinc ions. FIG. 6 shows the putative binding mode of DATA.QS.EuE. The EuE binding motif causes an extension of the linker and associated spatial shift of the QS linker, which impairs electrostatic interaction with the amino acids in the binding pocket. Subsequent in vitro assays confirmed the results of the docking analyses.

### Example 10b: Squaric Acid as Modulator of Excretion

Scheme 38 shows an example of an active ingredient conjugate or labeling precursor with a targeting vector for PSMA and a squaric acid group conjugated to the targeting vector.

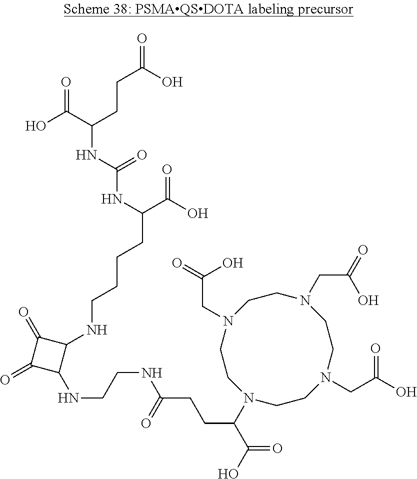 [SMILES: O=C(O)CCC(NC(=O)NC(CCCCNC1C(=O)C(=O)C1NCCNC(=O)CCC(C(=O)O)N1CCN(CC(=O)O)CCN(CC(=O)O)CCN(CC(=O)O)CC1)C(=O)O)C(=O)O]

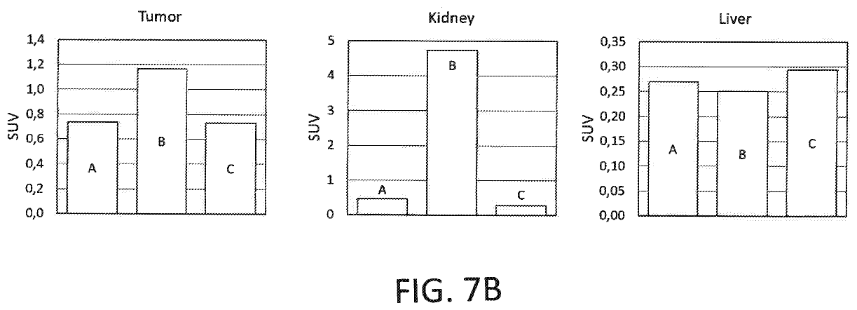

The conjugation of squaric acid (QS) to the PSMA Tracer reduces accumulation in the kidneys and the associated masking or disturbance of the PET signal from the adjacent prostate, which crucially improves sensitivity and reliability in the imaging diagnosis of prostate carcinoma by means of PET. FIGS. 7a and 7b show μPET images (60 min p.i.) Of [⁶⁸Ga]Ga.DOTA.QS.PSMA (A), [⁶⁸Ga]Ga-PSMA-11 (B) and [⁶⁸Ga]Ga-PSMA-617 (C) and a diagram with SUV values (standard uptake value: SUV) for tumor tissue, kidney and liver.

Scheme 39 shows a further QS derivative that has been tested in vivo in tumor-carrying animals.

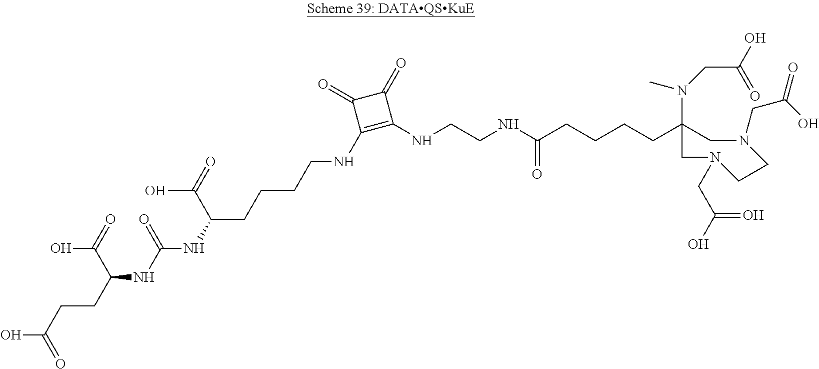 [SMILES: CN(CC(=O)O)C1(CCCCC(=O)NCCNc2c(NCCCC[C@H](NC(=O)N[C@@H](CCC(=O)O)C(=O)O)C(=O)O)c(=O)c2=O)CN(CC(=O)O)CCN(CC(=O)O)C1]

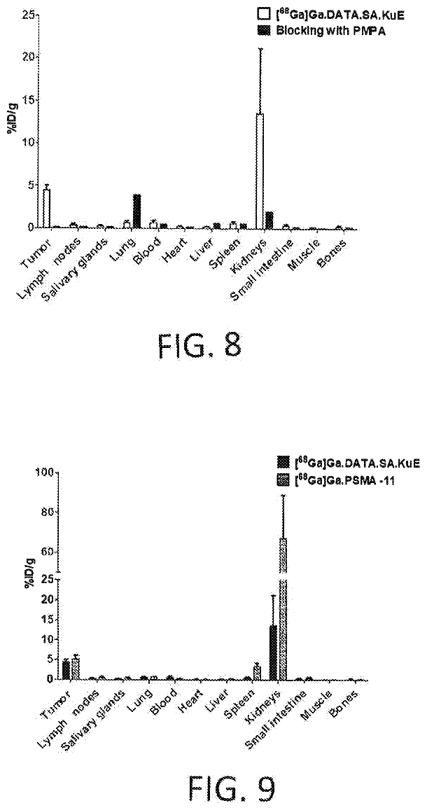

DATA.QS.KuE was labeled with ⁶⁸Ga and tested in vivo on LNCaP tumor-carrying Balb/c mice. FIG. 8 shows the accumulation of [⁶⁸Ga]-DATA.QS.KuE in the organs (biodistribution). The selectivity of binding was determined by means of competitive co-injection of the PSMA inhibitor PMPA. By way of comparison, FIG. 9 shows the biodistribution of [⁶⁸Ga]-PSMA-11.

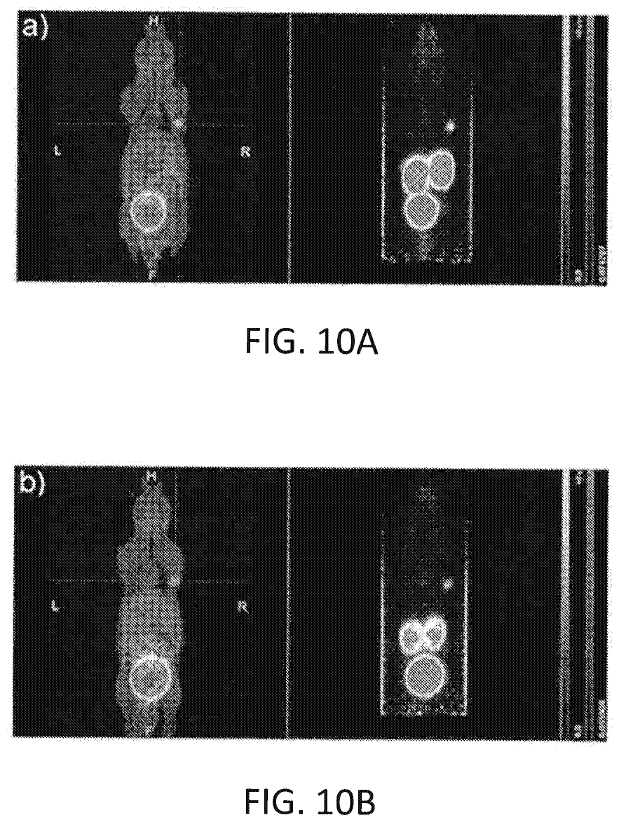

FIGS. 10a and 10b show the maximum-intensity projections from μPET studies with [⁶⁸Ga]-PSMA-11 and, respectively, [⁶⁸Ga]-DATA.QS.KuE in LNCaP tumor-carrying Balb/c.

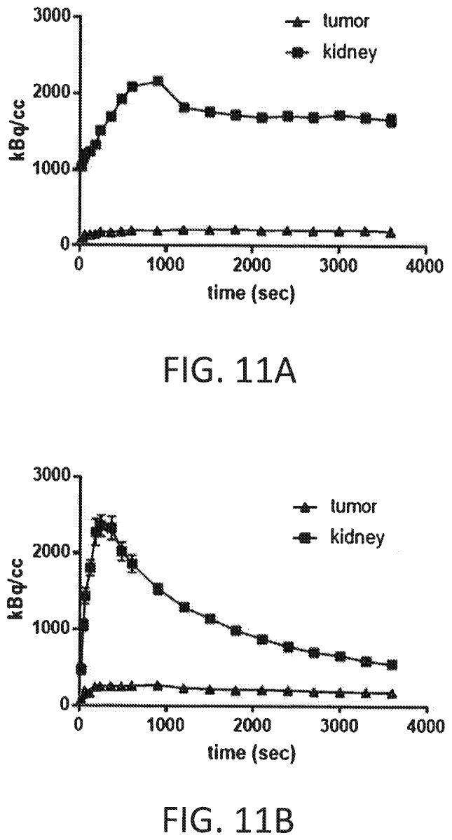

FIGS. 11a and 11b Showtime-activity curves of [⁶⁸Ga]-PSMA-11 and, respectively, [⁶⁸Ga]-DATA.QS.KuE. With approximately the same tumor enrichment, DATA.QS.KuE, by comparison with PSMA-11, shows considerably lower kidney exposure/dose. In the case of treatment with highly ionizing radionuclides, for example ¹⁷⁷Lu, rather than ⁶⁸Ga, DATA.QS.KuE enables a crucial reduction in nephrotoxicity.

### Example 11a: Evaluation of the In Vitro PSMA Binding Affinity of Selected Compounds and Compound Constituents

By means of a cell-based assay, the affinity of the target vector-linker units QS.KuE, QS.K.EuE and KuE with a lipophilic linker—analogously to PSMA-617—and the affinity of the substructures NH₂.DOTAGA.617.KuE and NH₂-DOTAGA.QS.KuE was determined. In addition, the PSMA affinity of the structure MMAE.ValCit.QS.617.KuE which is preferred in accordance with the invention (see scheme 30) was determined.

For the essay, LNCaP cells were pipetted into multiwell plates (Merck Millipore Multiscreen™). The compounds to be analyzed were each admixed with a defined amount or concentration of the reference compound ⁶⁸Ga[Ga]PSMA-10 with a known K_{d} value and incubated in the wells with the LNCaP cells for 45 min. After repeated washing, the cell-bound activity was determined. The inhibition curves obtained were used to calculate the IC₅₀ values and Kᵢ values reported in table 1.

TABLE 3
PSMA binding affinities

| Compound | IC₅₀ (nM) | Kᵢ (nM) |
|---|---|---|
| PSMA-617 | 15.1 ± 3.8 | 12.3 ± 3.1 |
| QS.KuE-TV linker unit | 35.9 ± 2.6 | 29.3 ± 2.1 |
| QS.EuE-TV linker unit | 17.2 ± 5.2 | 14.0 ± 4.2 |
| 617.KuE-TV linker unit | 21.5 ± 1.9 | 17.5 ± 1.5 |
| NH2.DOTAGA.617.KuE | 20.2 ± 3.6 | 16.5 ± 3.0 |
| [natGa]Ga-NH2.DOTAGA.617.KuE | 20.4 ± 9.4 | 16.8 ± 7.7 |
| [natLu]Lu-NH2.DOTAGA.617.KuE | 26.0 ± 4.7 | 21.4 ± 3.9 |
| NH2.DOTAGA.QS.KuE | 20.2 ± 3.5 | 18.1 ± 2.9 |
| DATA.QS.EuE | 386.0 ± 81.0 | 315.4 ± 66.2 |
| MMAE.ValCit.QS.617.KuE | 198.1 ± 1.9 | 161.9 ± 3.3 |

In order to determine non-specific binding, all compounds were additionally admixed with an excess of the PSMA inhibitor 2-PMPA (2-(phosphonomethyl)-pentanoic acid) and subjected to the same LNCaP assay—as described above.

Both the TV linker units and the chelator-TV linker units have similar affinity for PSMA to the reference compound PSMA-617. Accordingly, the use of QS as linker unit leads to an affinity comparable to the use of the peptidic PSMA-617. Neither coupling to the DOTAGA chelator nor labeling thereof with the radionuclides gallium-68 and lutetium-177 leads to any decrease in affinity.

The use of the binding unit EuE rather than KuE leads to a considerable deterioration in PSMA affinity. The results confirm the findings of the docking studies with regard to the unfavorable orientation of the EuE derivative in the PSMA binding pocket.

The coupling of the sterically demanding cytostatic MMAE and the ValCit linker and the TV linker unit QS.617.KuE leads to a distinct lowering of affinity.

### Example 7b: Determination of the Cytotoxic Action of the Dimeric Compound MMAE.ValCit.QS.617.KuE In Vitro

In a CellTiter Blue assay, LNCaP cells were incubated with the substance to be studied for 72 hours, and then the IC₅₀ of the compound was determined. Table 4 shows the IC₅₀ values of the compound MMAE.ValCit.QS.617.KuE which is preferred in accordance with the invention (scheme 30) compared to the pure active ingredient MMAE.

TABLE 4
Cytotoxic action in vitro

| Compound | IC₅₀ (nM) |
|---|---|
| MMAE | 0.29 ± 0.12 |
| MMAE.ValCit.QS.617.KuE | 32.2 ± 5.7 |

Although the inventive compound MMAE.ValCitQS.617.KuE shows somewhat lower cell cytotoxicity in vitro than the pure active ingredient MMAE, it is nevertheless in the lower nanomolar range.

1. A compound for dual nuclear-medical/cytotoxic theranostics having the structure 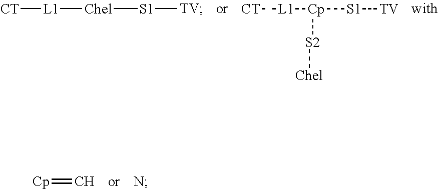
wherein
Chel is a radical of a chelator for the complexation of a radioisotope;
CT is a radical of a cytotoxic compound;
TV is a biological targeting vector;
L1 is a linker;
S1 and S2 are each a spacer.

2. A smart drug delivery system for dual nuclear-medical/cytotoxic theranostics, comprising
a first compound as claimed in claim 1 having the structure 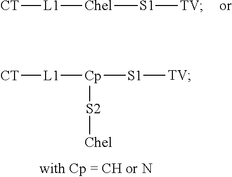
or
a second compound having the structure Chel-S-TV and a third compound having the structure CT-L-TV;
wherein, in the first, second and third compounds,
Chel is a radical of a chelator for the complexation of a radioisotope;
CT is a radical of a cytotoxic compound;
TV is a biological targeting vector;
L1 and L are each a linker;
S1, S2 and S are each a spacer.

3. A pharmaceutical kit for dual nuclear-medical/cytotoxic theranostics as claimed in claim 1, consisting of
a first vessel containing a first compound or a first carrier substance containing the first compound;
or
a second vessel containing a second compound or a second carrier substance containing the second compound,
and
a third vessel containing a third compound or a third carrier substance containing the third compound;
wherein the first compound has the structure 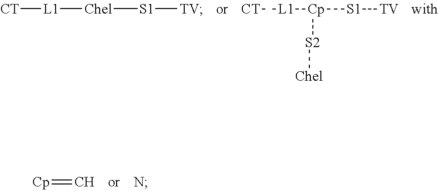
the second compound has the structure Chel-S-TV;
and the third compound has the structure CT-L-TV,
wherein
Chel is a radical of a chelator for the complexation of a radioisotope;
CT is a radical of a cytotoxic compound;
TV is a targeting vector selected from one of the structures [1] to [18] 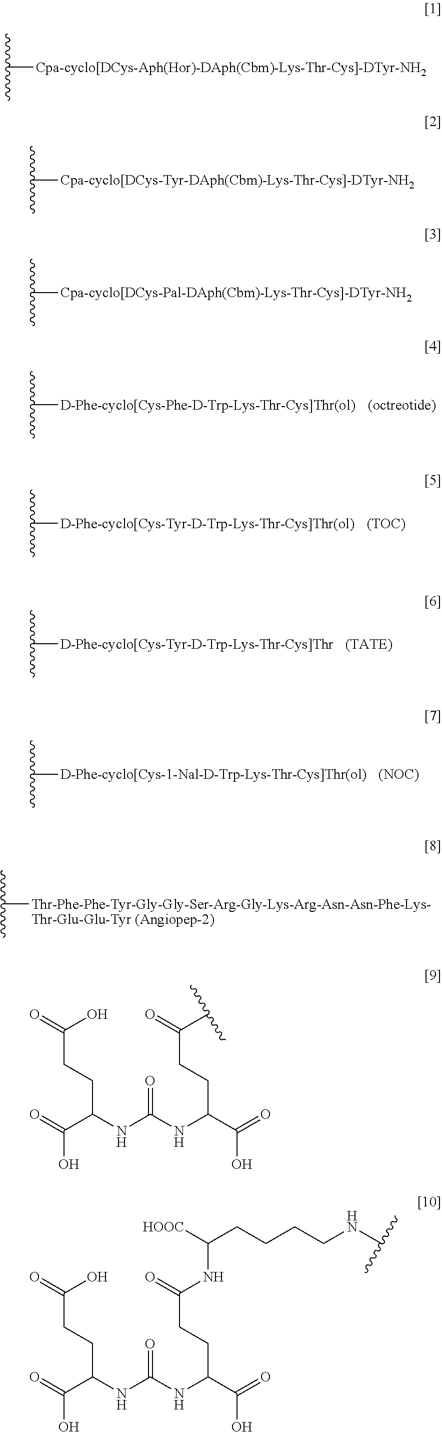 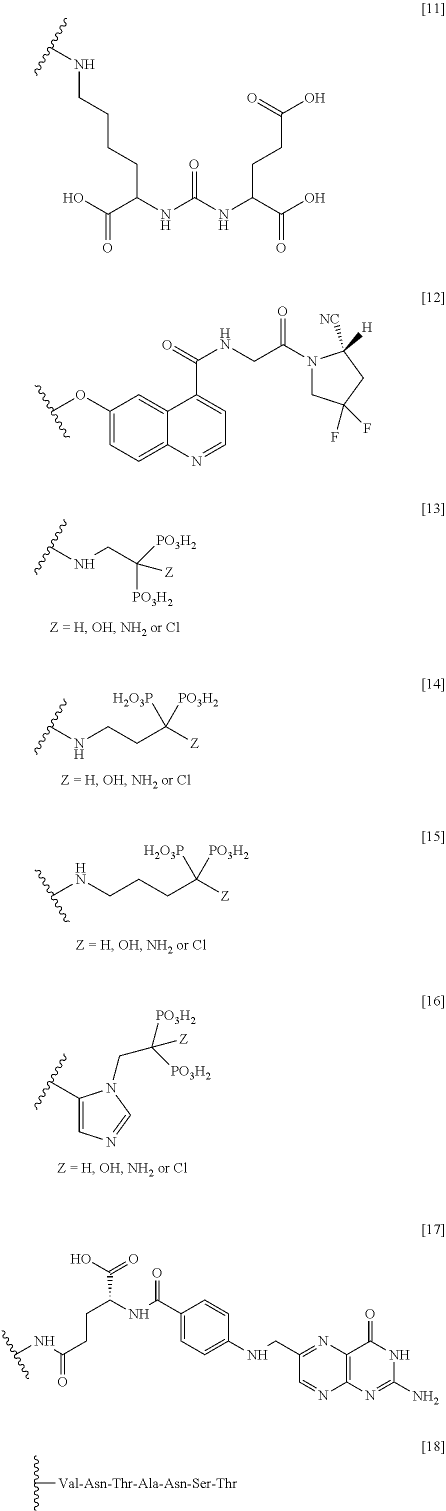
where the structures [1] to [8] and [18] denote amino acid sequences;
L and L1 independently have a structure selected from 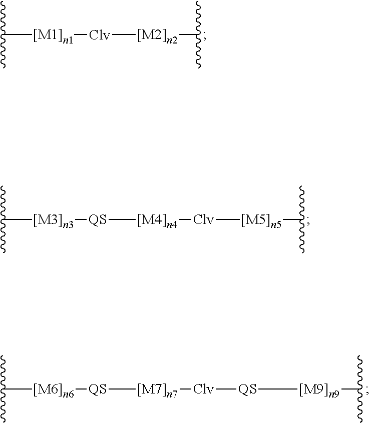
in which M1, M2, M3, M4, M5, M6, M7, M8 and M9 are independently selected from the group comprising amide, carboxamide, phosphinate, alkyl, triazole, thiourea, ethylene, maleimide radicals, —(CH₂)—, —(CH₂CH₂O)—, —CH₂—CH(COOH)—NH— and —(CH₂)ₘNH— with m=1, 2, 3, 4, 5, 6, 7, 8, 9 or 10;
n1, n2, n3, n4, n5, n6, n7, n8 and n9 are independently selected from the set of {0, 1, 2, 3, 4, 5, 6, 7, 8, 9, 10, 11, 12, 13, 14, 15, 16, 17, 18, 19, 20};
Clv is a cleavable group;
QS is a squaric acid radical 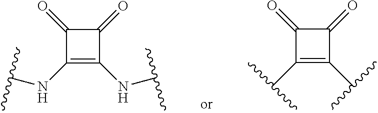 [SMILES: *Nc1c(N*)c(=O)c1=O ; *c1c(*)c(=O)c1=O]
S is the same as L (S=L); and/or
S, S1 and S2 independently have a structure selected from 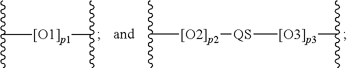
in which
O1, O2 and O3 are independently selected from the group comprising amide, carboxamide, phosphinate, alkyl, triazole, thiourea, ethylene, maleimide radicals, —(CH₂)—, —(CH₂CH₂O)—, —CH₂—CH(COOH)—NH— and —(CH₂)_{q}NH— with q=1, 2, 3, 4, 5, 6, 7, 8, 9 or 10; and
p1, p2 and p3 are independently selected from the set of {0, 1, 2, 3, 4, 5, 6, 7, 8, 9, 10, 11, 12, 13, 14, 15, 16, 17, 18, 19, 20}.

4. The radiopharmaceutical kit as claimed in claim 3, wherein CT is a radical of a cytotoxic compound selected from adozelesin, alrestatin, anastrozole, anthramycin, bicalutamide, bizelesin, bortezomib, busulfan, camptothecin, capecitabine, carboplatin, carzelesin, CC-1065, chlorambucil, cisplatin, cyclophosphamide, cytarabine (ara-C), dacarbazine (DTIC), dactinomycin, daunorubicin, dexamethasone, disulfiram, docetaxel, doxorubicin, duocarmycin A, duocarmycin B1, duocarmycin B2, duocarmycin C1, duocarmycin C2, duocarmycin D, duocarmycin SA, erismodegib, etoposide (VP-16), fludarabine, fluorouracil (5-FU), flutamide, fulvestrant, gemcitabine, goserelin, idarubicin, ifosfamide, L-asparaginase, leuprolide, lomustine (CCNU), mechlorethamine (nitrogen mustard), megestrol acetatr, melphalan (BCNU), menadione, mertansine, metformin, methotrexate, milataxel, mitoxantrone, monomethylauristatin E (MMAE), motesanib, maytansinoid, napabucasin, NSC668394, NSC95397, paclitaxel, prednisone, pyrrolobenzodiazepine, pyrvinium pamoate, resveratrol, rucaparib, S2, S5, salinomycin, saridegib, shikonin, tamoxifen, temozolomide, tesetaxel, tetrazole, tretinoin, verteporfin, vinblastine, vincristine, vinorelbine, vismodegib, α-chaconine, α-solamargine, α-solanine, or α-tomatine.

5. The radiopharmaceutical kit as claimed in claim 3, wherein the cleavable group Clv is selected from the group comprising

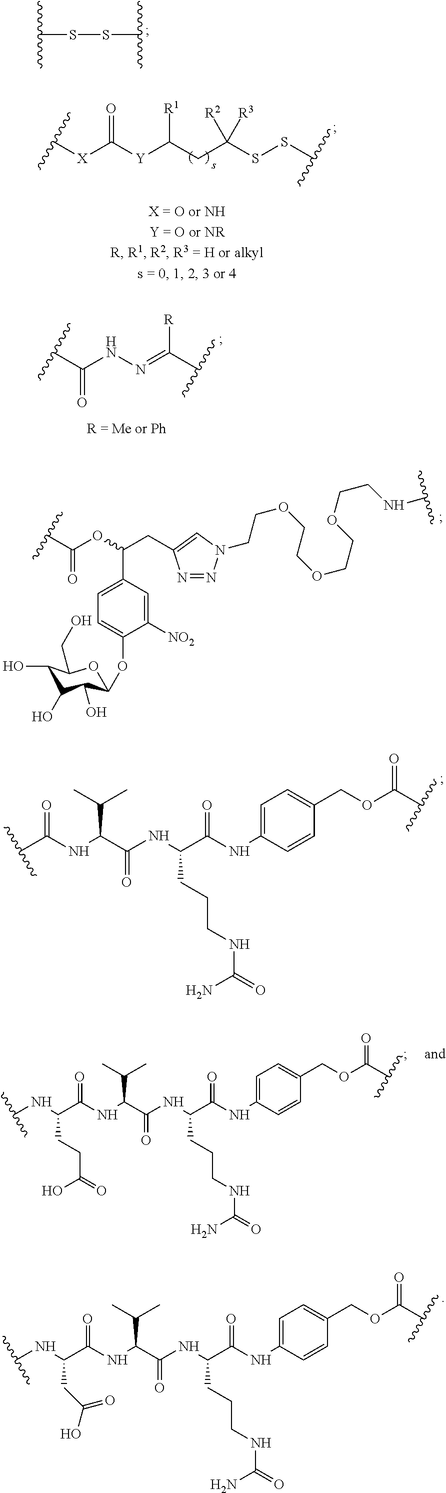

6. The radiopharmaceutical kit as claimed in claim 3, wherein the chelator Chel is selected from the group comprising H₄pypa, EDTA (ethylenediaminetetraacetate), EDTMP (diethylenetriaminepenta(methylenephosphonic acid)), DTPA (diethylenetriaminepentaacetate) and derivatives thereof, DOTA (dodeca-1,4,7,10-tetraaminetetraacetate), DOTAGA (2-(1,4,7,10-tetraazacyclododecane-4,7,10)-pentanedioic acid) and other DOTA derivatives, TRITA (trideca-1,4,7,10-tetraaminetetraacetate), TETA (tetradeca-1,4,8,11-tetraaminetetraacetate) and derivatives thereof, NOTA (nona-1,4,7-triaminetriacetate) and derivatives thereof, TRAP (triazacyclononanephosphinic acid), NOPO (1,4,7-triazacyclononane-1,4-bis[methylene(hydroxymethyl)-phosphinic acid]-7-[methylene(2-carboxyethyl)-phosphinic acid]), PEPA (pentadeca-1,4,7,10,13-pentaamine pentaacetate), HEHA (hexadeca-1,4,7,10,13,16-hexaamine hexaacetate) and derivatives thereof, HBED (hydroxybenzylethylene-diamine) and derivatives thereof, DEDPA and derivatives thereof, DFO (deferoxamine) and derivatives thereof, trishydroxypyridinone (THP) and derivatives thereof, TEAP (tetraazacyclodecanephosphinic acid) and derivatives thereof, AAZTA (6-amino-6-methylperhydro-1,4-diazepine-N,N,N′,N′-tetraacetate) and derivatives; SarAr (1-N-(4-aminobenzyl)-3,6,10,13,16,19-hexaazabicyclo[6.6.6]-eicosane-1,8-diamine) and salts thereof, (NH₂)₂SAR (1,8-diamino-3,6,10,13,16,19-hexaazabicyclo[6.6.6]eicosane) and salts and derivatives thereof, or aminothiols and derivatives thereof.

7. The radiopharmaceutical kit as claimed in claim 3, wherein the spacer S is the same as L (S=L).

8. The radiopharmaceutical kit as claimed in claim 3, wherein the first, second and third carrier substances are independently selected from the group comprising water, 0.45% aqueous NaCl solution, 0.9% aqueous NaCl solution, Ringer's solution (Ringer's lactate), 5% aqueous dextrose solution and aqueous alcohol solutions.

9. The radiopharmaceutical kit as claimed in claim 3, wherein the NOTA derivative is NOTAGA (1,4,7-triazacyclononane, 1-glutaric acid, 4,7-acetate), the DEDPA derivative is H₂DEDPA (1,2-[[6-(carboxylate-)pyridin-2-yl]methylamino]ethane), the trishydroxypyridinone derivative is YM103, and the AAZTA derivative is DATA ((6-pentanoic acid)-6-(amino)methyl-1,4-diazepine triacetate).
